(19) United States
(12) Patent Application Publication
(10) Pub. No.: US 2017/0198026 A1
(43) Pub. Date: Jul. 13, 2017

(54) Compositions And Methods For Treating Tumors And Immune Based Inflammatory Diseases

(71) Applicant: The Schepens Eye Research Institute, Inc., Boston, MA (US)

(72) Inventors: Joan Stein-Streilein, Boston, MA (US); Saimon Gordon, Boston, MA (US)

(21) Appl. No.: 15/316,790

(22) PCT Filed: Jun. 5, 2015
(86) PCT No.: PCT/US15/34524

§ 371 Date: Dec. 6, 2016

Related U.S. Application Data
(60) Provisional application No. 62/009,124, filed on Jun. 6, 2014.

(51) Int. Cl. C07K 14/72; C12N 15/113; C12N 5/0786; A61K 31/713; A61K 38/17; C07K 16/28; A61K 39/395

(52) U.S. Cl. CPC C07K 14/723; C07K 16/2896; C12N 15/1136; A61K 39/39558; A61K 31/713; A61K 38/1796; C12N 5/0645; C07K 2317/24; C12N 2310/14; C12N 2501/15

(57) ABSTRACT

This present invention provides compositions and methods for treating cancer and immune inflammatory disorders by modulating EMR2 signaling pathway.

### RELATED APPLICATIONS

This application claims the benefit of priority under 35 U.S.C. §119(e) to U.S. Provisional Application No. 62/009,124, filed Jun. 6, 2014, which is incorporated herein by reference in its entirety.

### FIELD OF THE INVENTION

The present invention relates to a compositions and methods for treating cancer and immune inflammatory diseases by modulating EGF-Like Module Containing, Mucin-Like, Hormone Receptor-Like 2 (EMR2) and its signaling pathway.

### BACKGROUND OF THE INVENTION

Tumors have usurped many of their host mechanisms for avoiding host defenses and immune responses. One of these tumor strategies is to set up adhoc immune privileged environments that are not dissimilar to the immune privileged environment of the eye. Thus, treating with immune cells alone may not get through the tumor regulatory mechanisms.

A number of approaches have been tried. For example, one approach is to expose dendritic cells to patient tumor antigens to generate a vaccine against a cancer. This approach is being used in treating hormone refractory prostate cancer (e.g., PROVENGE® (APC8015)). The second approach in clinical trials is the use of chimeric antigen receptor T-cell therapy. Patient T-cells are removed and genetically altered generating specific antigen receptors on their surface. The cells are multiplied and infused back into the patients where the T-cells can attack cancer cells. The third approach is to isolate T cells from tumors, expand the number of cells in culture and inject the cells back into the patient where the cells can attack tumor cells. However, these approaches, like others, do not address a fundamental problem, which is tolerance to tumor antigens that is limiting the development of immunotherapy for tumors.

Therefore, there is a need for a different approach that overcomes this difficulty.

### SUMMARY OF THE INVENTION

The invention provides a solution to this longstanding problem and provides an alternative to traditional immunotherapy for treatment of patients diagnosed with cancer and/or immune inflammatory disease.

Accordingly, the invention provides methods and compositions for treating tumor and/or immune based inflammatory disease. The composition includes an antagonist of a component of EGF-like module-containing mucin-like hormone receptor-like 2 (EMR2) signaling pathway, where the antagonist blocks the development of myeloid cells expressing EMR2, for example, regulatory T cells. The antagonist inhibits either the function or the expression of EMR2 or other components in EMR2 signaling pathway. The component is EMR2 or an EMR2 ligand (e.g., chondroitin sulfate). For example, the antagonist is an anti-EMR2 antibody or EMR2-binding fragment thereof. Alternatively, the antagonist is an siRNA against EMR2. The antagonist can also be a small molecule or a small peptide molecule.

Also provided is a method of treating or alleviating a symptom of cancer in a subject in need thereof by administering to the subject an effective amount of the composition described herein. Preferably, the cancer is a solid tumor cancer (such as sarcomas, carcinomas or lymphomas). For example, the cancer is brain and CNS cancer, kidney cancer, ovarian cancer, pancreatic cancer, lung cancer, breast cancer, colon cancer, prostate cancer, or a hematological cancer.

The method described above can also be combined with an autologous immune enhancement therapy, where the autologous immune enhancement therapy comprises administering to the subject an effective amount of autologous T immune cells. Preferably, the autologous immune enhancement therapy is performed after administration of the composition described herein to the subject.

Also provided herein is a method of inhibiting development or reducing population of myeloid cells expressing EMR2, by contacting a myeloid cell expressing EMR2 with the composition described herein. For example, the myeloid cell expressing EMR2 is a regulatory T cell. The regulatory T cell expresses CD4 and CD25. Alternatively, the regulatory T cell expresses CD8.

Further provided herein is an isolated antibody against EMR2 or F4/80 or a fragment thereof. The antibody can be a monoclonal antibody, a polyclonal antibody, a chimeric antibody or a single-chain antibody. Alternatively, the antibody can be a humanized monoclonal antibody. For example, the antibody binds to an epitope located in the stalk region of EMR2.

As used herein, an “isolated” or “purified” nucleotide or polypeptide is substantially free of other nucleotides and polypeptides. Purified nucleotides and polypeptides are also free of cellular material or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified nucleotides and polypeptides is one that is at least 90%, 91%, 92%, 93%, 94%, 95%, 98%, 99%, or 100% (w/w) of the desired oligosaccharide by weight. Purity is measured by any appropriate standard method, for example, by column chromatography, thin layer chromatography, or high-performance liquid chromatography (HPLC) analysis. The nucleotides and polypeptides are purified and used in a number of products for consumption by humans as well as animals, such as companion animals (dogs, cats) as well as livestock (bovine, equine, ovine, caprine, or porcine animals, as well as poultry). “Purified” also defines a degree of sterility that is safe for administration to a human subject, e.g., lacking infectious or toxic agents.

Described herein also includes an isolated peripheral blood monocyte engineered to express EMR2. Any known method available in the art can be utilized to express EMR2 in peripheral blood monocytes.

Such engineered peripheral blood monocytes can be used to suppress inflammation in a subject in need thereof by administering to the subject an effective amount of the isolated peripheral blood monocytes. The isolated peripheral blood monocytes may be incubated with TGF-beta (e.g., TGF-beta 1 or TGF-beta 2) and a tissue extraction prior to the administration. The extraction is isolated from the diseased tissue where the inflammation occurs. In some instances, the subject is suffering from a CD4-mediated inflammatory disease. For example, the CD4-mediated inflammatory disease is diabetes, Lupus erythematosus, an allergy, rheumatoid arthritis, multiple sclerosis or Crohn's disease. In some instances, the inflammation is ocular inflammation.

Further provided herein is a method of treating or alleviating a symptom of an ocular immune inflammatory disease in a subject in need thereof by administering to the subject an effective amount of an agonist of EMR2. For example, the ocular immune inflammatory disease comprises dry eye syndrome, uveitis, corneal ulcer, ocular cicatricial pemphigoid, Mooren's ulcer, scleritis, scleritis or immunogenic conjunctivitis.

Also provided herein is a method of treating a CD4-mediated inflammatory disease in a subject in need thereof by a) isolating a sample comprising a peripheral blood monocyte cell from the subject; b) incubating the sample with TGF-beta (e.g., human TGF-beta 1 or 2) and one of the following: an antigen that is specific for the diseased tissue and an extract of the diseased tissue; and c) administering the incubated sample to the subject, thereby treating the CD4-mediated inflammatory disease. For example, the CD4-mediated inflammatory disease is diabetes, Lupus erythematosus, an allergy, rheumatoid arthritis, multiple sclerosis or Crohn's disease.

“Combination therapy” used herein embraces the administration of the composition as described above in further combination with other biologically active ingredients and non-drug therapies (e.g., surgery or radiation treatment).

Alternatively, combination therapy includes the administration of the composition as described above in further combination with an autologous immune enhancement therapy. For example, the autologous immune enhancement therapy comprises administering to the subject an effective amount of autologous T immune cells.

As used herein, a “subject in need thereof” is a subject having been diagnosed with a disorder, or a subject having an increased risk of developing such disorder relative to the population at large. A subject in need thereof can have a precancerous condition. Preferably, a subject in need thereof has been diagnosed with cancer. A “subject” includes a mammal. The mammal can be e.g., a human or appropriate non-human mammal, such as primate, mouse, rat, dog, cat, cow, horse, goat, camel, sheep or a pig. The subject can also be a bird or fowl. Preferably, the mammal is a human.

The term “an effective amount”, as used herein, refers to an amount of a composition to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is an immune-based inflammatory disease.

An effective amount of a composition is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped.

As used herein, “treating” or “treat” describes the management and care of a patient for the purpose of combating a disease, condition, or disorder and includes the administration of a composition described herein to alleviate the symptoms or complications of a disease, condition or disorder, or to eliminate the disease, condition or disorder. The term “treat” can also include treatment of a cell in vitro or an animal model.

As used herein, the term “alleviate” is meant to describe a process by which the severity of a sign or symptom of a disorder is decreased. Importantly, a sign or symptom can be alleviated without being eliminated. The administration of compositions of the invention may or can lead to the elimination of a sign or symptom, however, elimination is not required. Effective dosages should be expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

As used herein the term “symptom” is defined as an indication of disease, illness, injury, or that pathological state. Symptoms are felt or noticed by the individual experiencing the symptom, but may not easily be noticed by others. Others are defined as non-health-care professionals.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

These and other capabilities of the invention, along with the invention itself, will be more fully understood after a review of the following figures, detailed description, and claims. All references cited herein are hereby incorporated by reference.

### BRIEF DESCRIPTION OF THE DRAWINGS

FIG. 1 is diagram of synergistic processes of the ocular immune system. The ocular immune system includes multi-faceted defense mechanisms. Physical barriers, anti-inflammatory mediators, suppressive cell and humoral immunologic factors are among many contributors to ocular immune privilege (used with permission, Stein-Streilein, 2006).

FIG. 2A is a diagram showing molecular structures of human EGF-TM7. FIG. 2B is a diagram showing molecular structures of three largest EGF-TM7 molecules. FIG. 2C is a diagram showing human EMR2 and its sequential homology with CD97 and EMR3.

FIG. 3 is a diagram showing a process of generating in vitro tolerogenic APC (human) cells.

FIGS. 4A-4D are bar graphs showing phenotypic profile of tolerogenic APC. Each bar shows the mean±SEM of three repeats. Y-axis represents percent of cells positive for each marker given the treatment condition. FIG. 4A is a bard graph that shows the expression of CD14 on all cells treatment groups. FIG. 4B is a bar graph that shows the expression of ILT3 on all cells treatment groups. FIG. 4C is a bar graph that shows the expression of EMR2 on all cells treatment groups. FIG. 4D is a bar graph that shows the expression of PD-L1 on all cells treatment groups.

FIGS. 5A-5D are graphs showing flow cytometric APC marker profiles. FIG. 5A is a graph showing the flow cytometric profiles of CD14. FIG. 5B is a graph showing the flow cytometric profiles of ILT3. FIG. 5C is a graph showing the flow cytometric profiles of PD-L1. FIG. 5D is a graph showing the flow cytometric profiles of EMR2. In each of FIGS. 5A-5D the four markers were stained on the human tolerogenic APCs. Red corresponds to the unstained control; blue, cells treated with antigen alone; green, cells treated with antigen and TGFβ2. TGFβ2 alone showed similar fluorescence intensities as antigen alone (data not shown). ILT3, PD-L1, EMR2 were analyzed on a gated CD14 population to ensure majority monocytic cells for analysis. Y-axis depicts percentage of cells out of total monocyte population. X-axis shows the fluorescence intensity of surface marker staining to the unstained control.

FIG. 6 is a diagram showing procedure of generating in vitro human anterior chamber associated immune deviation (ACAID).

FIGS. 7A-7C are scatter plots showing function of induced T regulatory cells. Intracellular staining for FoxP3, IFN-γ, and IL-10 of CD4+ gated lymphocytes post co-culture with tolerogenic APC. FIG. 7A is a bar graph of CD4+ lymphocytes stained for surface CD25 and intracellular FoxP3. Each donor was repeated in duplicate. Y-axis represents percent total of cells positive for both CD25 and FoxP3 with regards to each treatment condition. FIGS. 7B and 7C are bar graphs of CD4+CD25+ lymphocytes stained for intracellular IFN-γ (FIG. 7B) or IL-10 (FIG. 7C). Y-axis represents percent total of cells positive for IFN-γ or IL-10.

FIGS. 8A-8C are plots showing flow cytometric dot plots of intracellular protein production. All quadrant gates were created on unstained untreated single color control populations. Quadrant II, as referenced in the text, is the upper right area of each graph which reflects populations that immunostained positive for both allophycocyanin and phycoerythrin fluorochromes. FIG. 8A shows cytometric dot plots of CD4+ lymphocyte cells, with TGFβ, and with (right panels) or without (left panels) antigen. Y-axis measures the CD25 marker by allophycocyanin (APC-A) fluorochrome, X-axis measures the intracellular FoxP3 marker by phycoerythrin (PE-A) fluorochrome. Both axes measure fluorescence intensity. Quadrant II (upper right corner) of each graph represents the CD4+CD25+ FoxP3+ population. FIG. 8B shows cytometric dot plots of CD4+CD25+ lymphocyte cells, with TGFβ, and with or without antigen. Y-axis measures intracellular IFN-γ cytokine by allophycocyanin fluorochrome, X-axis measures intracellular IL-10 cytokine by phycoerythrin fluorochrome. Both axes measure fluorescence intensity. FIG. 8B is a representative plot of decreased IFN-γ production in the presence of antigen. FIG. 8C shows cytometric dot plots of CD4+CD25+ lymphocyte cells, with TGFβ, and with or without antigen. Y-axis measures intracellular IFN-γ cytokine by allophycocyanin fluorochrome, X-axis measures intracellular IL-10 cytokine by phycoerythrin fluorochrome. Both axes measure fluorescence intensity. FIG. 8C is a representative plot of increased IL-10 production in the presence of antigen. Control samples for untreated and antigen alone conditions not shown.

FIG. 9 is a bar graph showing FoxP3 expression of T cells treated with soluble chondroitin sulfate. Intracellular staining of FoxP3, surface staining of CD25 on CD4+ gated lymphocytes post co-culture with tolerogenic APC. Y-axis represents percent of total lymphocytes positive for FoxP3 and CD25. Donor was repeated in duplicate.

FIGS. 10A-10B are plots showing flow cytometric EMR2 readout and dot plot post-soluble chondroitin sulfate treatment. Quadrant II, as referenced in the text, is the upper right area that reflects populations that immunostained positive of both APC-A and PE-A. FIG. 10A is a flow cytometry plot showing APC that were cultured with antigen and with (right panels) or without (left panels) TGFβ. Both conditions received 1.5 mg/mL of soluble chondroitin sulfate from shark cartilage. Red corresponds to the unstained control, blue corresponds to the APC experimental conditions. Y-axis represents percentage of cells out of total monocyte population, X-axis represents fluorescence intensity of EMR2 staining compared to the unstained control. FIG. 10B is a flow cytometric dot plot showing TT alone vs TT and TGFβ with both conditions receiving 1.5 mg/mL of soluble chondroitin sulfate. Cells gated are CD4+ lymphocytes; Y-axis measures CD25 by allophycocyanin fluorochrome and X-axis measures FoxP3 by phycoerythrin fluorochrome. Quadrant gates were created on unstained untreated single color controls. Control samples for untreated and TGFβ alone are not shown.

FIG. 11 is a diagram showing in vivo assay for Treg suppression.

FIG. 12 shows Treg suppression of DTH in vivo.

FIG. 13 shows results of interfere with the generation of Treg cells by blocking EMR2.

FIG. 14A is a bar graph showing DTH response in humanized SCID mice. Each bar is mean±SEM of 3-5 animals per treatment condition. Y-axis shows the change in ear swelling in millimeters. X-axis shows experimental condition of mice in each group. FIG. 14B is a bar graph showing intracellular IFNg and IL-10 production of T cells. Flow cytometric results of CD4+CD25+ lymphocytes in co-culture with APC with or without 1b5. We see a significant abrogation in IL-10 production of cells in co-culture with 1b5-treated APCs as well as an increase in IFNg production. Each bar shows mean+/−SEM of three repeats.

FIG. 15 is a bar graph showing results of testing Treg function in vivo in suppressing DTH. Ear thickness was measured after injecting mice with Treg cells with or without EMR2 blockage.

FIG. 16 is an alignment of amino acid sequence of human EGF-TM7 receptors. EGF domains, the GPS motif and the seven hydrophobic transmembrane segments are indicated. An arrowhead shows the predicted processing site within the GPS motif. Inner and outer borders of overlap between the transmembrane segments, found in the differences, are given. Positions of conserved amino acids are indicated by asterisks for identical residues.

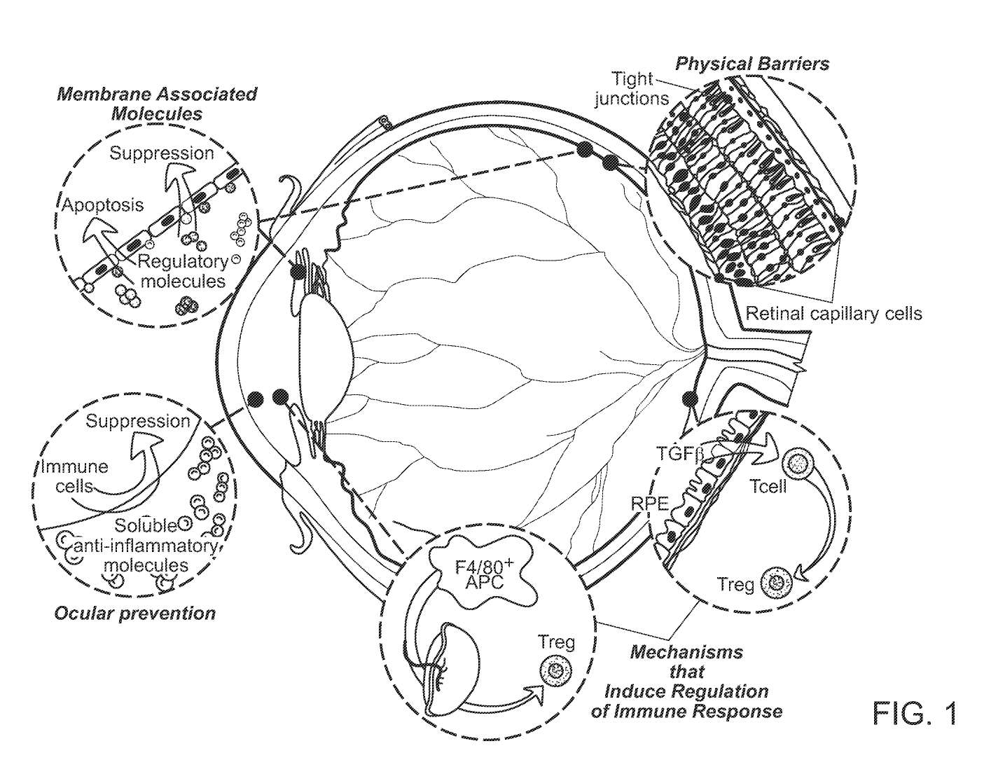

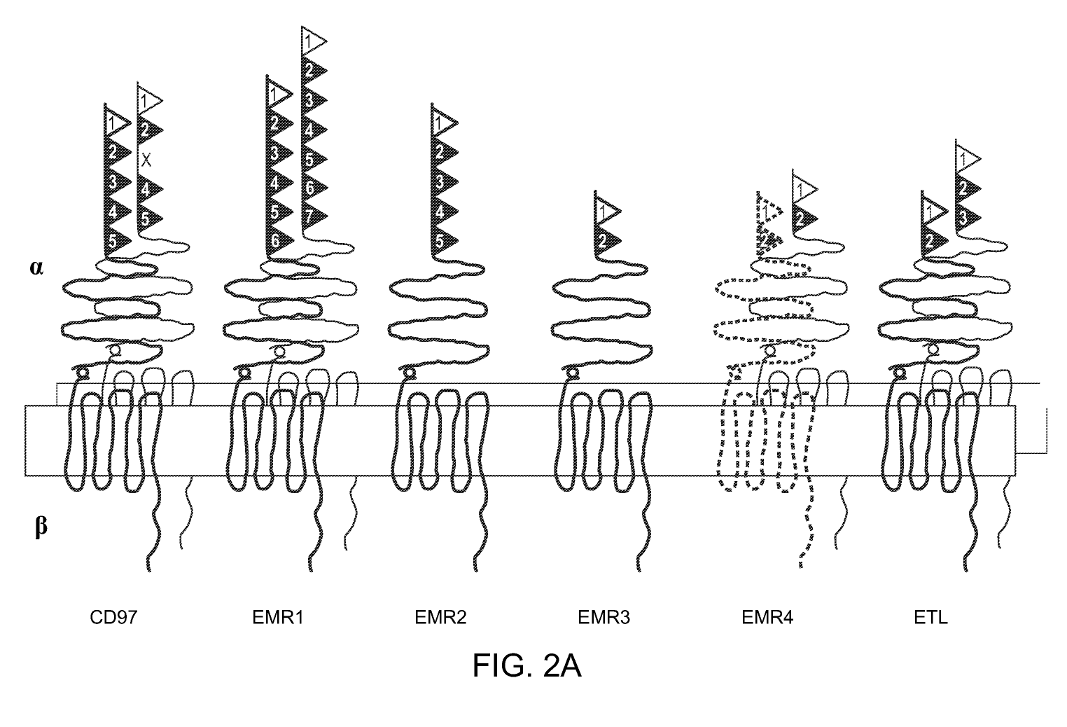

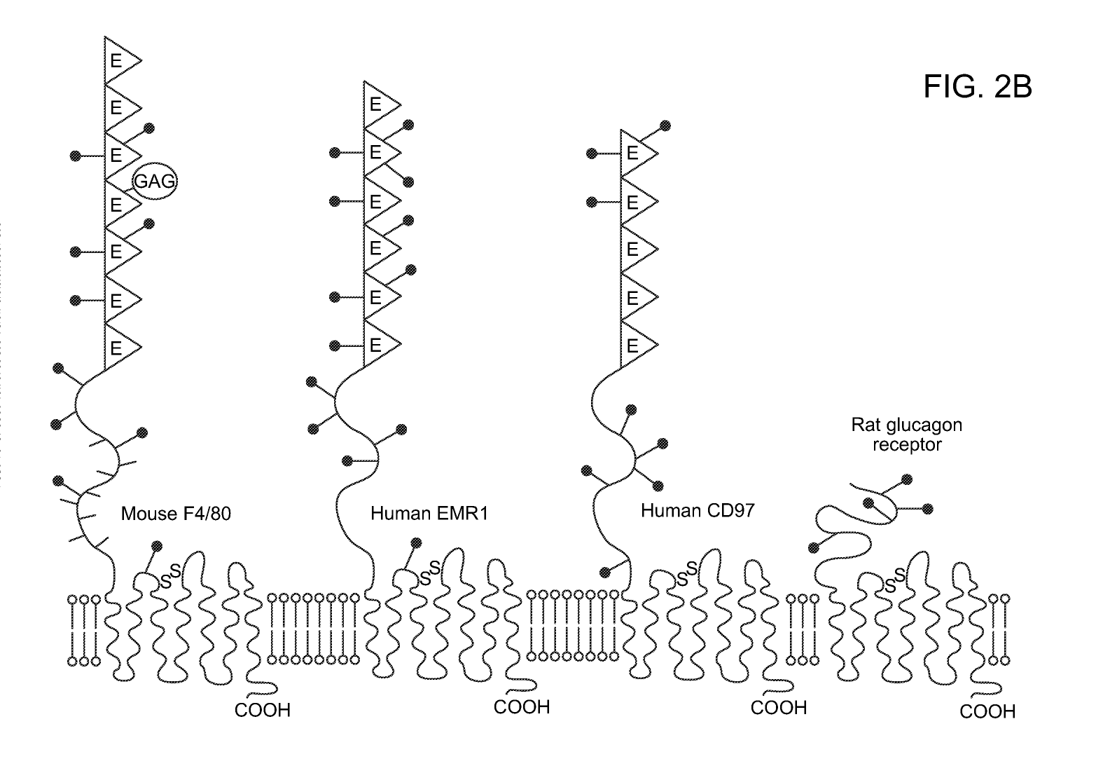

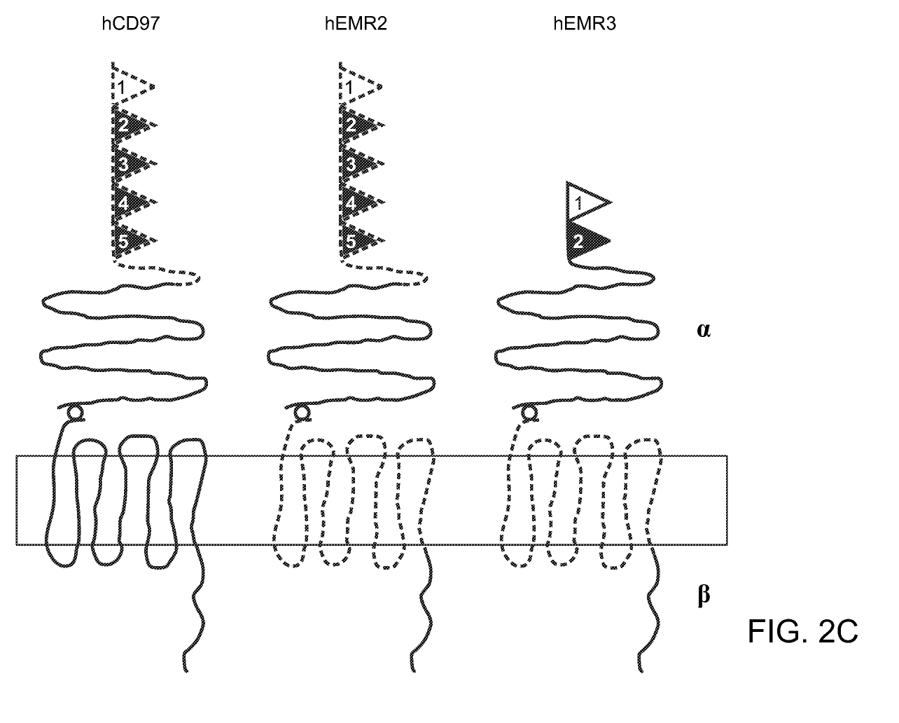

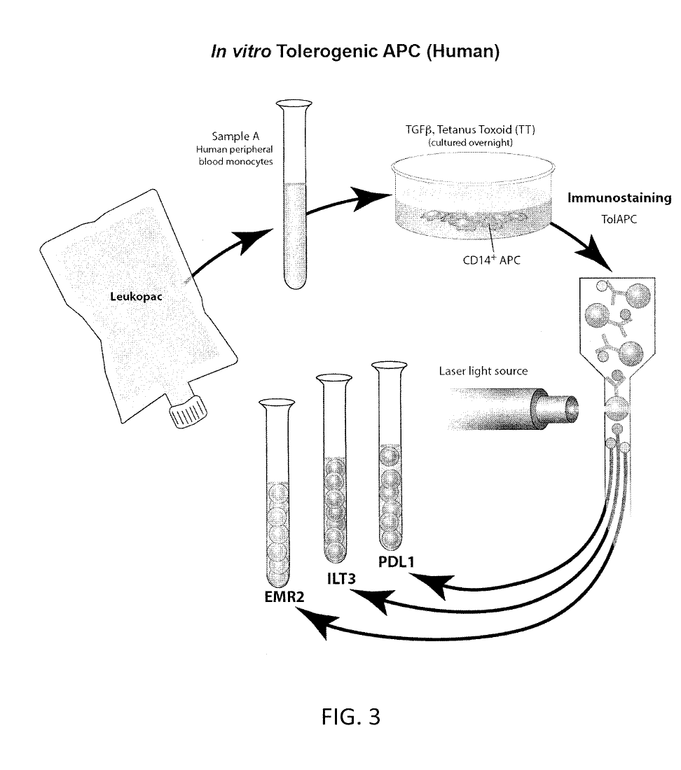

### DETAILED DESCRIPTION OF THE INVENTION

Tumors have usurped many of their host mechanisms for avoiding host defenses and immune responses. One of these tumor strategies is to set up adhoc immune privileged environments that are not dissimilar to the immune privileged environment of the eye. Thus, treating with immune cells alone may not get through the tumor regulatory mechanisms.

A hallmark of the immune privileged environment is the macrophage that may present the tumor antigens to T cells and support their differentiation into T regulatory cells. A T regulatory cell is able to control an immune cell capable of targeting the tumor antigens. The macrophages themselves may also have suppressor characteristics that kill, or anergize the immune cell, directly.

The data presented herein shows that in the mouse both the tolerogenic macrophage and the suppressor macrophage express F4/80 protein, which is necessary to induce T regulatory cells in both ocular and oral tolerance. As presented herein, by identifying the F4/80 equivalent in the human (EMR2), a tool to control and disrupt the tumor's regulatory mechanisms has been developed. The results demonstrated that antagonists to the EMR2 molecules interfere with the development of T regulatory cells in human cells.

This disclosure relates to discovery that modulating EMR2 signaling pathway can regulate the development of T regulatory cells.

The following materials and methods were used to generate the data described herein.

Collection of Human Blood Samples

Blood samples were purchased from the Massachusetts General Hospital Blood Transfusion Service donor pool. Human donor protocols were considered exempt and were in compliance as defined by Massachusetts Eye and Ear Infirmary Institutional Review Board procedures and in approval with Partners Subcommittee on Human Studies

Cell Isolation

Peripheral blood mononuclear cells (PBMC) were collected from anti-coagulated Leukopacs and washed twice with RPMI-1640 (Lonza, Basel, Switzerland), supplemented with 1 mM sodium pyruvate (Invitrogen, Carlsbad, Calif.), 0.1 mM non-essential amino acids (Invitrogen, Carlsbad, Calif.), 100 U/mL Penicillin/Streptomycin (Invitrogen, Carlsbad, Calif.), and 10% fetal bovine serum (FBS) (Atlanta Biologicals, Flowery Branch, GA). Monocytes were enriched by depletion of non-monocyte cells with Negative Monocyte Isolation Kit II (Miltenyi Biotec, Auburn, Calif.). Monocyte purity (˜90-95%) was assessed by fluorescence-activated cell sorting (FACS) and analyzed for the constitutive CD14 monocyte surface marker (BD Pharmingen, San Diego, Calif.).

Generation of Tolerogenic APC

Isolated cells were plated on sterile petri dishes (BD Falcon, Franklin Lakes, N.J.) in serum-free culture medium, RPMI-1640 supplemented with Glutamax (Invitrogen, Carlsbad, Calif.), 1 mM sodium pyruvate, 0.1 mM non-essential amino acids, 100 U/mL Penicillin/Streptomycin, 10 mM HEPES (Invitrogen, Carlsbad, Calif.), Insulin/Transferrin/Selenium (ITS) (Invitrogen, Carlsbad, Calif.), and 0.1% BSA (Sigma-Aldrich, St. Louis, Mo.). Cells were cultured with or without 1 ng/mL porcine TGFβ2 (R&D Systems, Minneapolis, Minn.) for at least six hours. Cells were then pulsed with or without tetanus toxoid (TT) antigen (List Biological Laboratories, Campbell, Calif.) in serum-free media and incubated overnight in 37° C., 5% CO2.

APC were analyzed by flow cytometry to confirm the expression of myeloid and tolerogenic markers. CD14 (constitutive on monocyte-derived cells), Immunoglobulin-like transcript 3 (ILT3, cell surface immunoglobulin that co-ligates to APC stimulatory receptors to downregulate activation signals (Cella et al., 1997)), Programmed cell death 1 ligand 1 (PD-L1, well-known immunosuppressive ligand), and EMR2 were measured. Monoclonal antibodies to ILT3 and PD-L1 were purchased from eBioscience (San Diego, Calif.) and the monoclonal antibody to EMR2 was purchased from R&D Systems (Minneapolis, Minn.).

Induction of T Regulatory Cells

Tolerogenic APC were washed twice with serum-free media to remove all of the exogenously added TGFβ. Cells were then cultured with an autologous T cell sample, obtained by Human T cell Enrichment Columns (R&D Systems, Minneapolis, Minn.). Antigen is then reintroduced to the cell co-culture prior to incubation at 37° C., 5% CO2 for five days. Following the incubation period, the cells were detached from the dishes by incubating with cold 2 mM EDTA solution in phosphate buffered saline (PBS) for 10-15 minutes. Co-culture cells were collected and immunostained for CD4 (T helper cell marker), CD25, and FoxP3 (in conjunction, regulatory T cell markers). In parallel, cells were tested for functionality by measuring intracellular production of IFN-γ (pro-inflammatory cytokine) and IL-10 (anti-inflammatory cytokine). All antibody markers and intracellular staining kit were purchased from eBioscience.

Blocking EMR2 Ligand Binding

Monocytes were cultured as outlined above. Soluble chondroitin sulfate from shark cartilage (Sigma-Aldrich, St. Louis, Mo.) was used to bind to EMR and block signaling at a concentration of 1.5 mg/mL. CD25 and FoxP3 expression as well as intracellular cytokine functionality was tested.

Flow Cytometric Analysis

Flow cytometric analysis was conducted.¹¹ Intracellular cytokine staining was performed to evaluate T cell activation. Conjugated mouse monoclonal antibodies specific for the following determinants were used: CD4 (clone OKT4; BioLegend, San Diego, Calif.), CD8a (clone RPA-T8; BioLegend), CD137 (clone 4B4-1; BD Biosciences, San Diego, Calif.), CD154 (clone TRAP1; BD Biosciences), and IFN-γ (clone B27; BD Biosciences). Appropriate isotype controls were included in each analysis.

Data Analyses

Flow cytometry data was collected on a BD LSR II via BD FACSDiva software. Flow cytometry data was analyzed using FlowJo software (Tree Star, Ashland, Oreg.). All data were compiled and statistically analyzed using GraphPad Prism (GraphPad Software, La Jolla, Calif.). For statistical analyses, *p≦0.05, **p≦0.01, ***p≦0.001, ****p≦0.0001 Differences between two groups were considered significant at p≦0.05.

Surface Markers on Human Tolerogenic APC

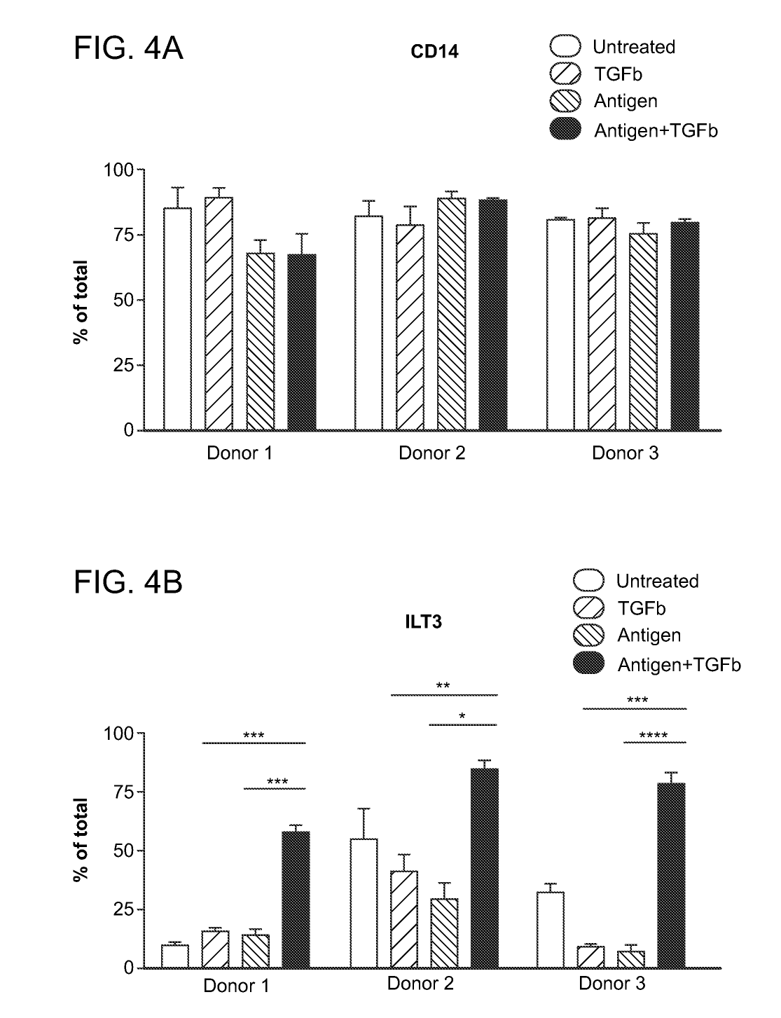

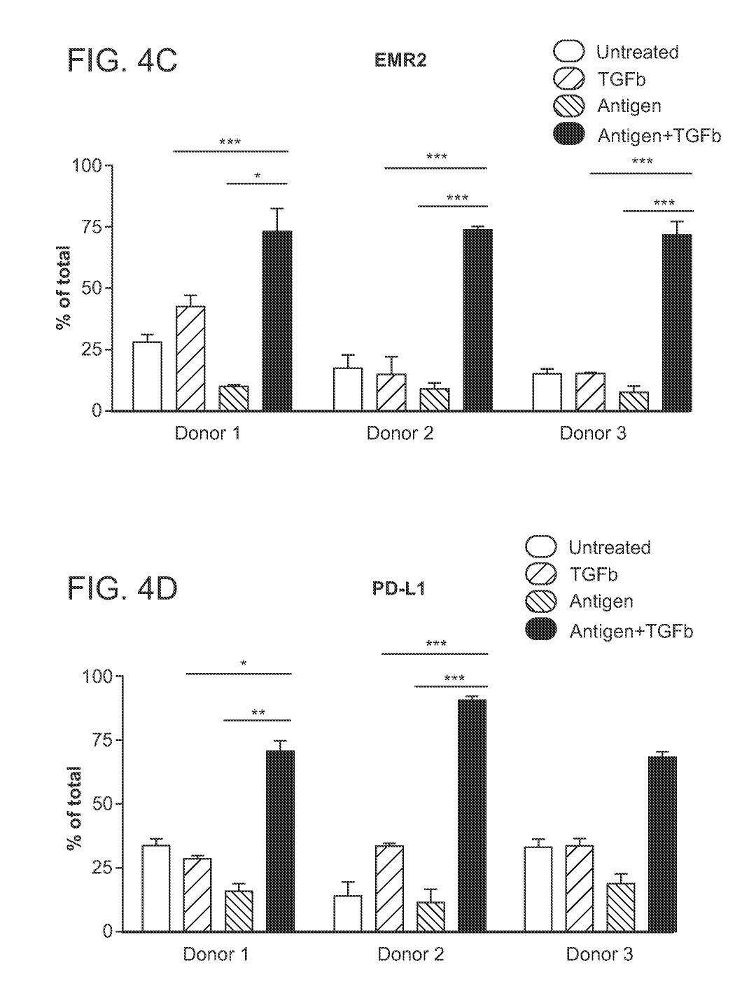

When treated with aqueous humor factors, namely TGFβ2, mouse F4/80 macrophages acquire the ability to induce T regulatory cells. When treated with TGFβ2, human APCs upregulate inhibitory co-receptors as opposed to classical co-stimulatory molecules, such as CD40 (data not shown). Here, we compared and contrasted the surface markers expressed on APCs post-exposure to TGFβ2 and antigen. CD14 remained constitutively expressed on all cell treatment groups (FIG. 4A). PD-L1, an inhibitory co-receptor, and ILT3, a novel immunosuppressive molecule, were significantly (p≦0.05) upregulated in the presence of both TGFβ2 and antigen when compared to antigen or TGFβ2 alone (FIG. 4D, FIG. 4B). The potential F4/80 analog, EMR2, was also upregulated (FIG. 4C). Significance was assessed by one-way ANOVA for each cell surface marker. A difference between two groups was considered significant if p≦0.05.

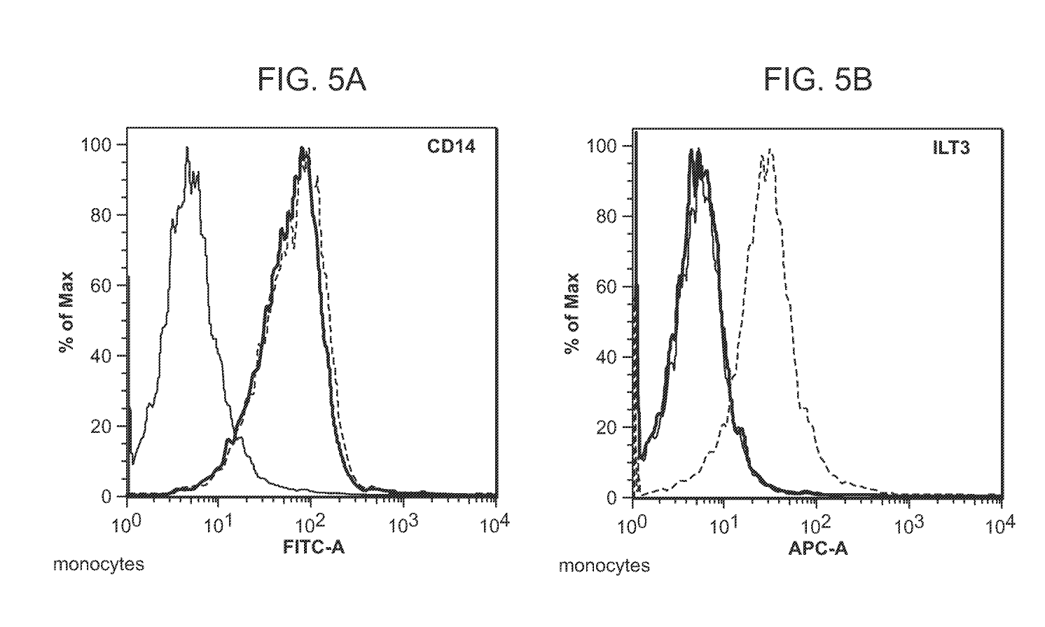

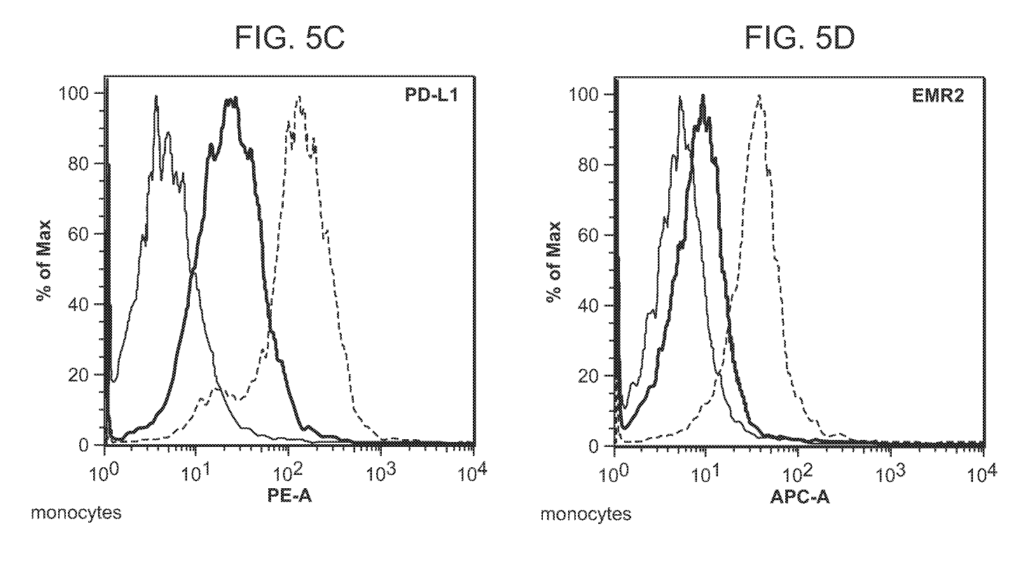

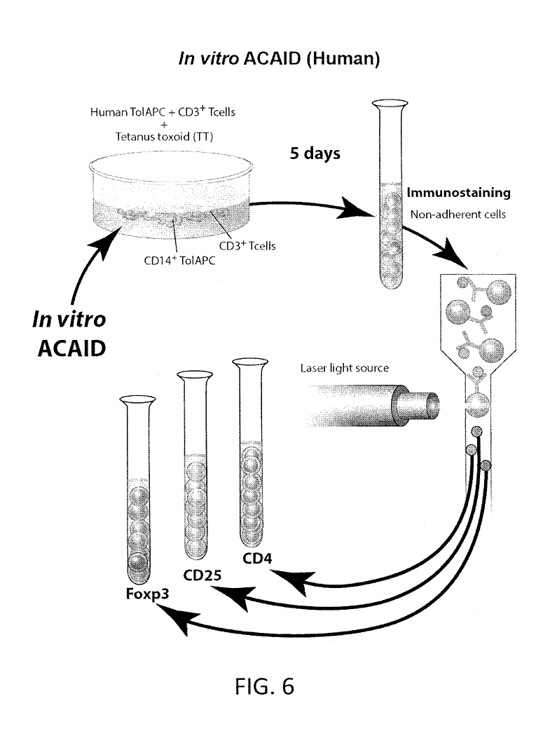

Flow cytometric data showed differences in surface marker expression in comparison to unstained control (FIGS. 5A-D).

Induced T Regulatory Cells

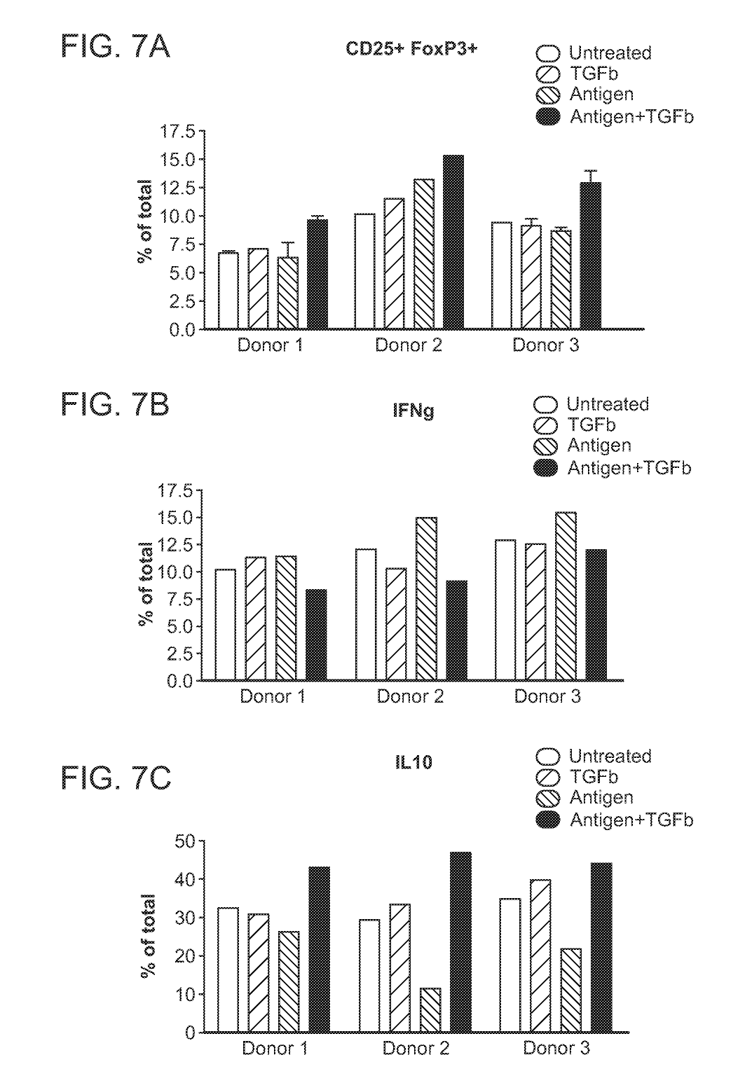

Post antigen inoculation into the anterior chamber of the mouse, F4/80+ APCs travel from the eye to the marginal zone of the spleen to facilitate cell aggregations. The interactions of cells within the aggregates induce T regulatory cells. To recreate the tolerance inducing environment for human cells, we essentially created a “spleen-in-a-dish” in which tolerogenic APCs were co-cultured with whole peripheral lymphocytes from the same blood sample. These lymphocytes were collected on a human T cell column in which unwanted cells attach to immunoglobulin or anti-immunoglobulin coated glass beads within the column. The resulting elution contained an enriched CD3+(a universal T cell marker) population. In the co-cultures, tolerogenic APC induced a population of CD25+ FoxP3+ lymphocytes. These non-adherent cells were collected and confirmed by surface staining the CD4 marker and analyzed for CD25 and FoxP3 (FIG. 7). T regulatory cells also increased production of the anti-inflammatory cytokine, IL-10 and decreased production of the pro-inflammatory cytokine, IFN-γ, also shown by FIGS. 5A-D.

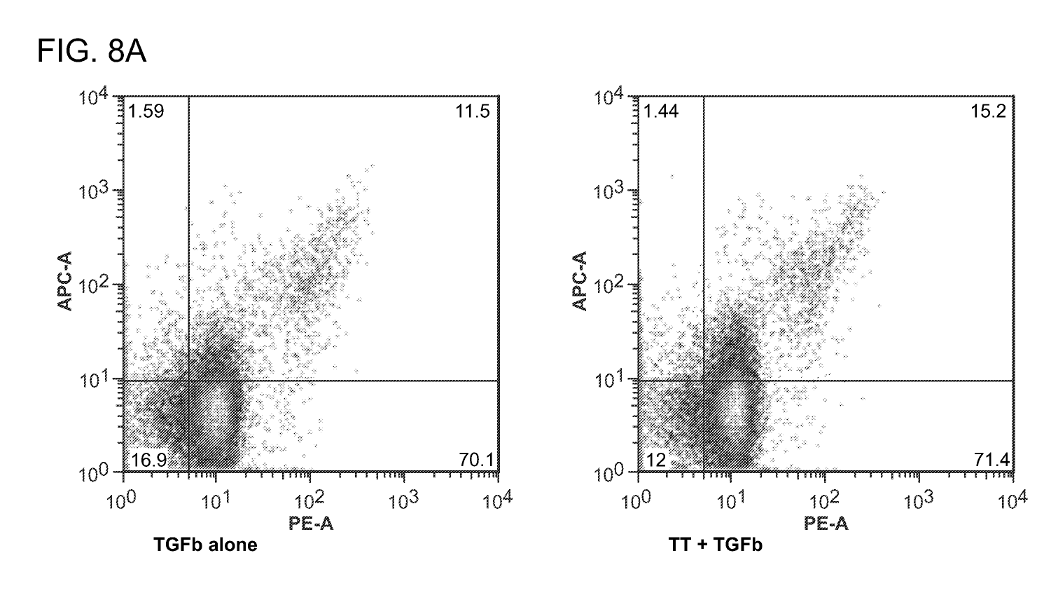

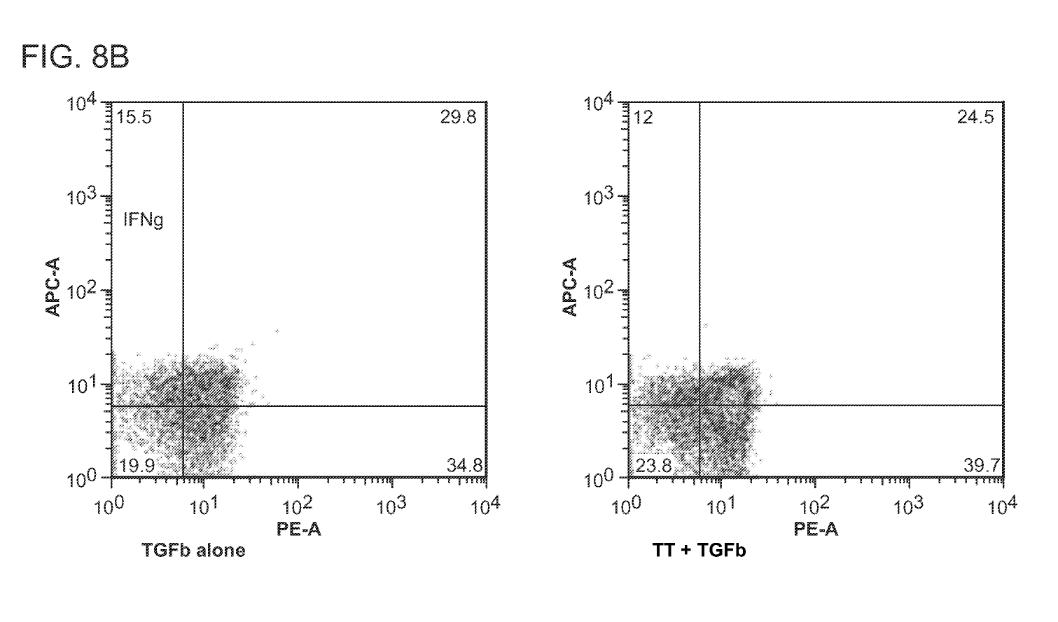

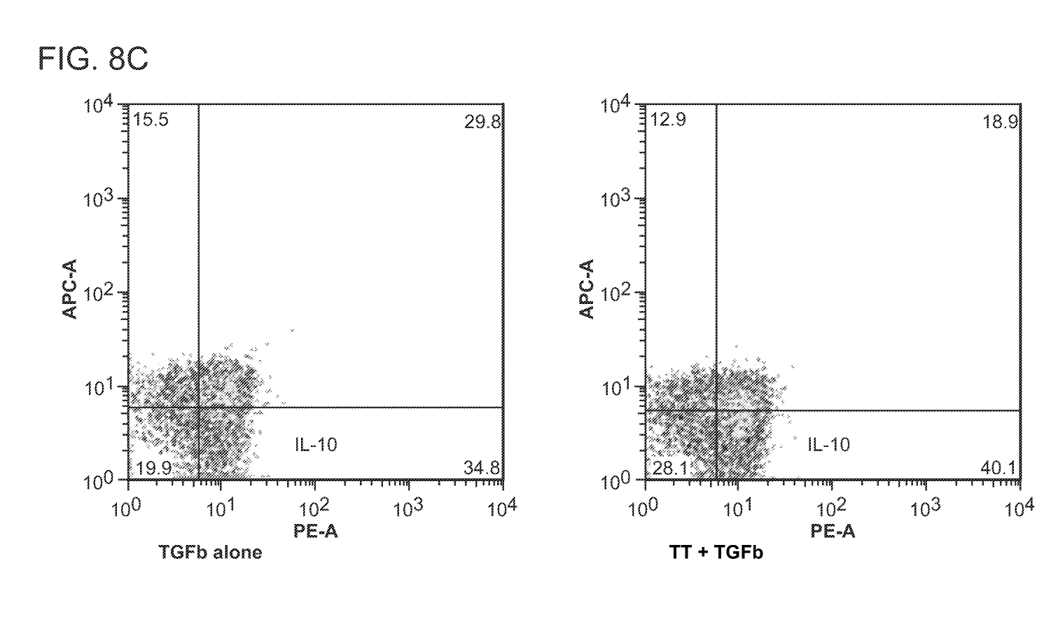

Flow cytometric analysis showed an increase of cells expressing both CD25 and FoxP3 from the cultures that contained the tolerogenic APC (FIG. 8A). In the presence of TGFβ and antigen, the number of cells producing intracellular IL-10 increased and the number of cells producing intracellular IFN-γ decreased (FIG. 8B, FIG. 8C).

Since our organ of interest is the eye, and the eye is protected by a blood-ocular barrier, we have used serum-free culture medium in all of our experiments. Also, serum contains known inhibitors of TGFβ. Previously tested by the Stein-Streilein group, serum interferes with the signaling of many immunosuppressive factors in the aqueous humor. Thus, each experiment was done without serum. However, once the tolerogenic APCs are generated, the induction of T regulatory cells can occur in complete medium because signals that lead to T regulatory generation involve cell-cell contact and are not affected by the presence of serum. Thus, some experiments were conducted in complete medium for the latter portion of the APC-lymphocyte co-culture.

Blocking EMR2 Ligand Binding

It has been found that the common ligand for EMR2 is chondroitin sulfate glycosaminoglycans (GAG) (Stacey et al., 2003). To mediate cell interactions, EMR2 binds to chondroitin sulfate GAGs on the opposing cell. To block EMR2 signaling and the induction of T regulatory cells, we added soluble chondroitin sulfate to the cell culture. Saturating the culture with soluble chondroitin sulfate competitively binds up available EMR2 molecules and subsequently block signaling from the APC to the lymphocytes.

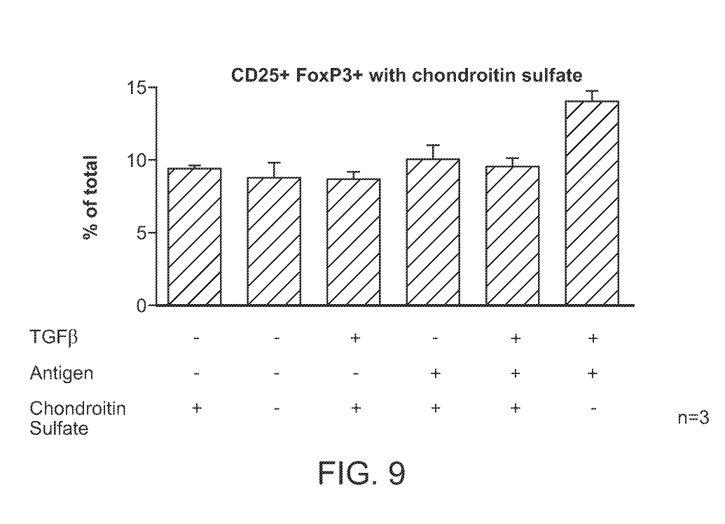

We treated APC with soluble chondroitin sulfate (1.5 mg/mL) in the same conditions listed above. By using a dose curve, we found 1.5 mg/mL was the ideal concentration for saturating EMR2 receptors. Post co-culture with lymphocytes, we repeated surface and intracellular staining of lymphocytes to check for differences in FoxP3+ cells (FIG. 9).

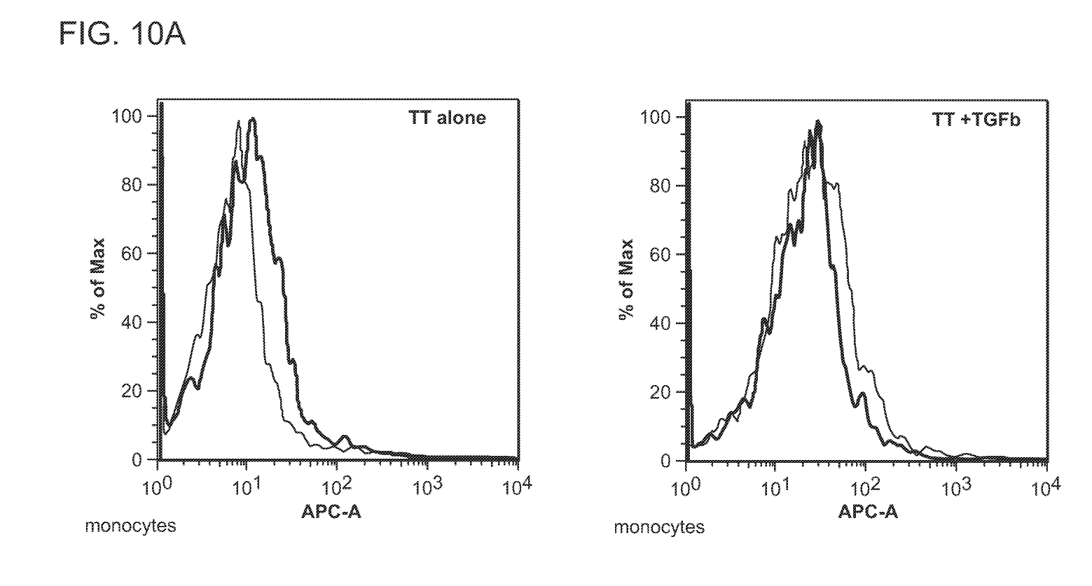

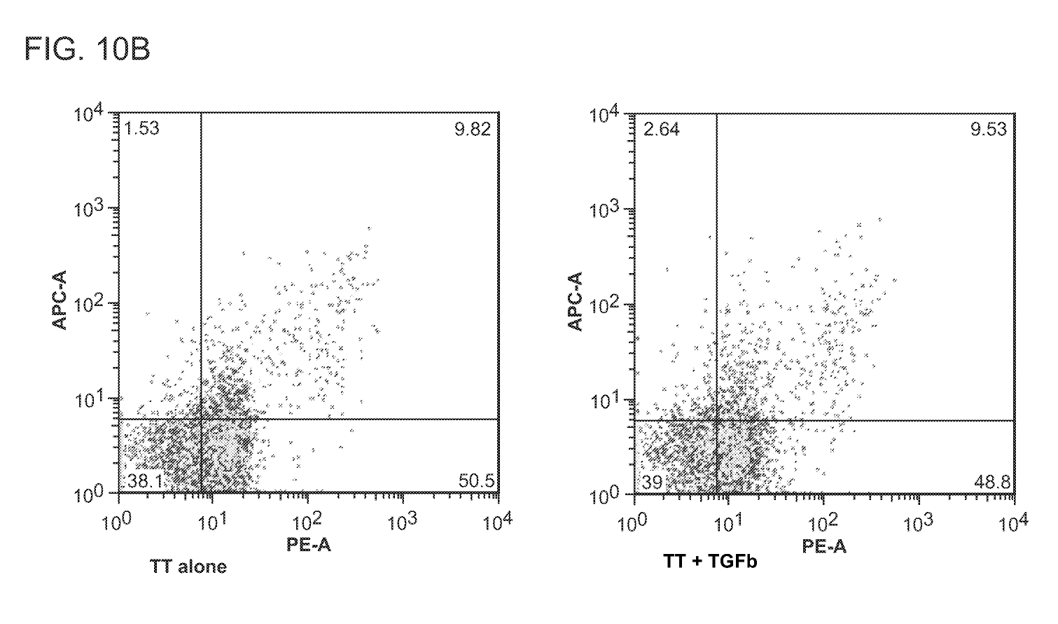

FIG. 10 shows flow cytometry readouts of the effect of using soluble chondroitin sulfate in the APC culture. In both FIG. 9 and FIGS. 10A-B, we see no increase in CD25+ FoxP3+ cell populations when treated with antigen and TGFβ.

In Vivo Model

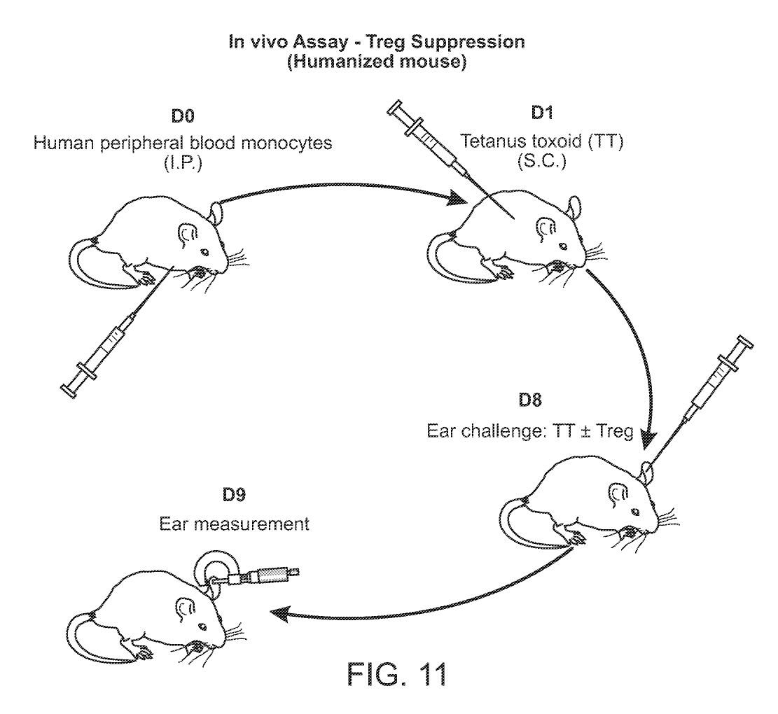

Once T regulatory cells were induced by our generated tolerogenic APCs, we began using NOD SCID mice to implement an in vivo model (FIG. 11). SCID mice are unable to undergo somatic recombination of their T cell receptor genes, thus they are unable to form unique genetic sequences on their T cell receptors that recognize a bounty of antigenic proteins. As a result, these mice lack functional and mature T and B cells. In these mice, we applied our experimental conditions by adding fresh human PBMC into the animal system. We immunized the animal against the antigen of interest. Seven days later, the animal was challenged with the same antigen with or without autologous T regulatory cells generated in vitro. The following day, ear-swelling representative of the DTH response, was measured. With human cells, we were able to induce ACAID in an immunocompromised mouse with tetanus toxoid.

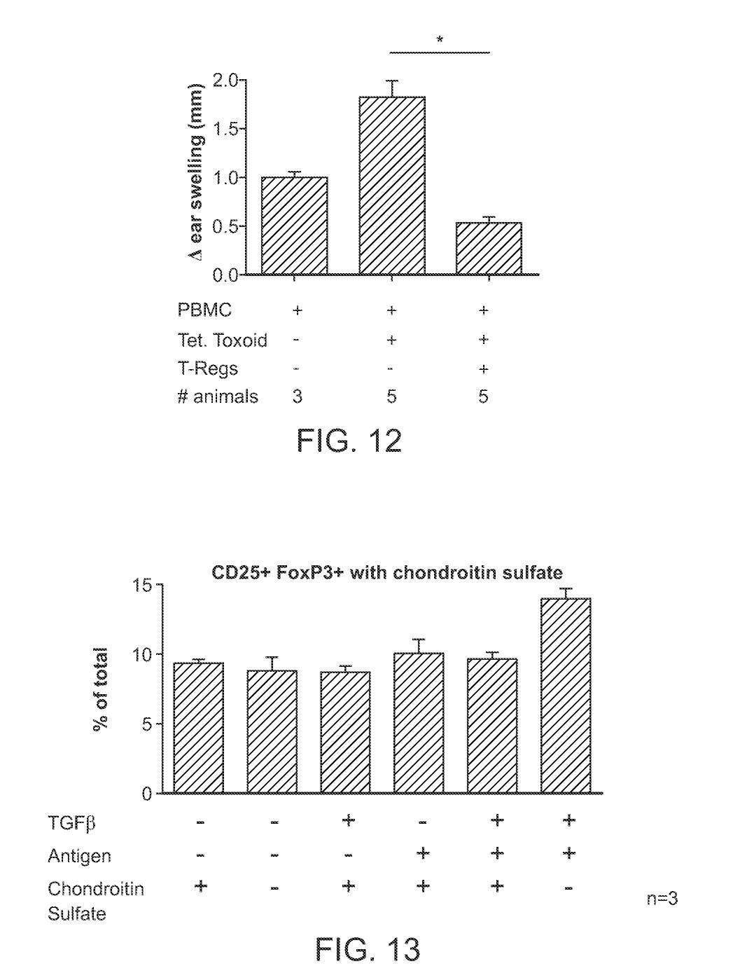

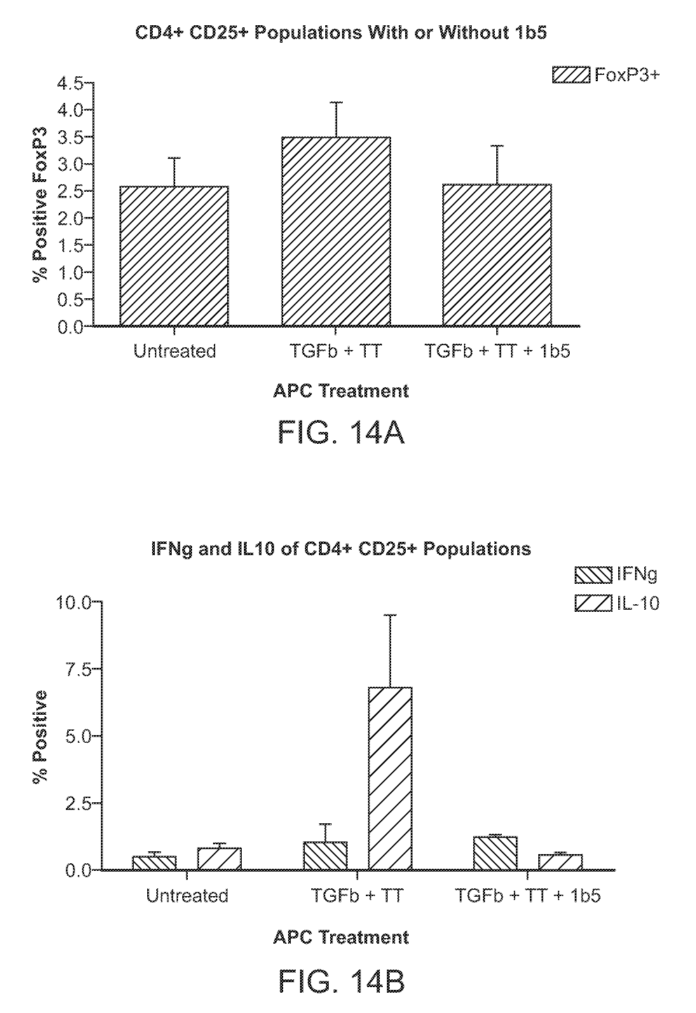

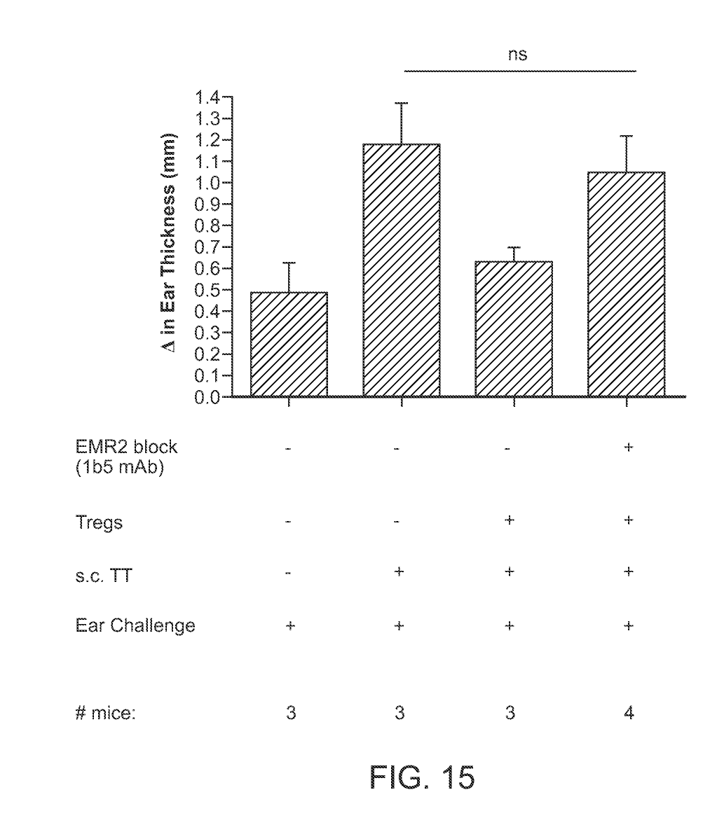

Compared to the control condition in which no T regulatory cells and no antigen were added, a large swelling response with the addition of TT antigen was observed. When TT antigen in the presence of autologous T regulatory cells was added to the animal, a significant decrease of swelling was observed. Thus, the in vitro generated T regulatory cells from EMR2+ APCs were able to suppress DTH inflammation in the mouse ear. See FIG. 12.

Immune privilege is a trait unique to certain parts of the body that are critical to life. In other areas exposed to the external environment or foreign antigens of the external environment, tolerance is necessary in order to keep excessive inflammation in check. The more we understand how the body regulates immune responses and induces tolerance, the possibility of manipulating characteristics of innate and adaptive immunity for therapeutic purposes becomes more plausible.

Human Tolerogenic APC

We determined that like F4/80 in the mouse, EMR2 is upregulated in human tolerogenic APCs post-treatment with aqueous humor-type immunosuppressive factors, namely TGFβ. Different from tolerogenic APCs, professional APCs present antigen and initiate T cell activation and proliferation. These two cell types differ phenotypically most notably in their co-stimulatory machinery. Professional APCs consist of dendritic cells, macrophages, and some B-cell types. Of the many types of dendritic cells, those myeloid cells that participate in T cell activation express CD11c and CD1a on their surface, which are classic dendritic cell markers. The professional macrophages express Mac-3 and CD11b. Both cell types constitutively display CD14, which indicate that they are of monocytic origin as well as the obligatory MHC class II and CD40 for co-stimulation of the T cell. In the delayed type hypersensitivity response, both will secrete IL-12.

In immune privilege, tolerogenic APCs are of interest. Upon interaction with T cells, these APCs will induce the regulatory T cell phenotype and no T cell activation and expansion will occur. On the tolerogenic APCs, similar surface markers are present such as CD40 and MHC class II. However, these APCs express inhibitory molecules that compete with co-stimulatory molecules to prevent T cell activation. These inhibitory molecules include PD-L1 and ILT3, which downregulate or counter activating signals. In the presence of upregulated inhibitory molecules, EMR2 is also upregulated. Thus, EMR2 may be a marker for human tolerogenic APCs. While we did not explicitly test for other classic macrophage or dendritic cell markers listed above, these cells were constitutively CD14 positive and differentiated into tolerogenic APCs by showing upregulated inhibitory molecules that would be expected on suppressive monocytic cells interacting with T cells.

Our finding that EMR2 is upregulated on these tolerogenic APCs is novel. As an adhesion G-protein coupled receptor, EMR2 is a member of the EGF-TM7 subfamily and found on cells of myeloid origin. F4/80, also a member of the EGF-TM7 subfamily, is upregulated on mouse macrophages in the same experimental conditions with TGFβ and antigen. As the F4/80 analog, the upregulation of EMR2 indicated that it functions in a synonymous manner to F4/80. Both are adhesion molecules capable of binding to glycosaminoglycans and we postulate that EMR2 has a significant role in cell-cell interactions. Through adhesion or signaling, EMR2 on the tolerogenic APCs may facilitate cell clustering with other T cells, B cells, and NKT cells to induce a T regulatory cell population, much like the role of F4/80

Functions of Human Tolerogenic APCs

Second, we showed that a population of CD4+CD25+ FoxP3+ lymphocytes increased post-culture with tolerogenic APC. The increase of this lymphocyte population in the mouse requires the presence of F4/80 on tolerogenic macrophages. When resting lymphocytes come into contact with F4/80 macrophages, the macrophages induce a peripheral lymphocyte population into the T regulatory phenotype, CD25+ FoxP3+. Much like the mouse, human tolerogenic APCs with upregulated EMR2 induced a T regulatory cell population that were able to suppress inflammation due to a specific antigen in vivo.

The significance of these inducible T regulatory cells is that they perpetuate tolerance to an antigen of interest in the periphery. There are two types of T regulatory cells, natural and induced. While natural T regulatory cells develop naturally within the thymus through T cell receptor affinity selection, inducible ones do not and are subject to a multitude of conditions outside the thymus. Development of extrathymic inducible T regulatory cells can occur within instances of inflammation in which effector T cells as well as regulatory T cells are developed simultaneously in a primary immune response. However, in conditions sans inflammation, inducible T regulatory cells can develop by antigen presentation from tolerogenic APCs. It is this latter condition in which our APCs induced other autologous peripheral lymphocytes into a population of CD25+ FoxP3+ cells. Thus, by priming our tolerogenic APCs with an antigen of interest, we induced tolerance in peripheral lymphocytes to become inducible T regulatory cells. In a living organism, the significance of an increased peripheral inducible T regulatory cell population indicates a systemic tolerance to that antigen from aqueous humor-derived tolerogenic APCs. This corroborates to the previously established mouse model and is the first step in translating tolerance and the mechanisms of immune privilege from the mouse to the human.

EMR2 and its Ligand, Chondroitin Sulfate Glycosaminoglycans (GAGs)

Lastly, we determined that blockade of EMR2 with a soluble ligand specific to EMR prevents the induction of CD4+CD25+ FoxP3+T regulatory cells. When F4/80 is blocked on mouse macrophages, they fail to produce T regulatory cells. When we blocked EMR2 ligand binding on human tolerogenic APCs, they similarly failed to produce T regulatory cells. From this, EMR2 is required for human tolerogenic APCs to induce a CD4+CD25+ FoxP3+T regulatory cell population. Without EMR2, peripheral tolerance cannot be induced to the antigen of interest because these T regulatory cells are not there to suppress inflammatory activity against that antigen. Thus the adhesion or signaling between EMR2 from the APC and its glycosaminoglycan ligand on peripheral lymphocytes is necessary in order to produce T regulatory cells that uphold tolerance around the tumor or throughout the body.

This discovery opens up the possibility of manipulating mechanisms of immune privilege for future therapies. It is possible that EMR2 is a marker for tolerogenic APCs in the human. As a suppressor molecule, it is likely to be found in areas of the body displaying immune privilege. From a pathological standpoint, these are likely to be areas with neoplastic growth. As our immune system recognizes tumor cells and abnormal growths of tissue, the presence of molecules like EMR2 help the tumors survive by participating in tolerance induction and evading immune surveillance. Blocking EMR2 and its subsequent signaling could potentially assist self immune defenses against the tumor.

In contrast, hyperinflammation is a hallmark of many different diseases. As inflammatory products are damaging to cells and tissues within the area, immune regulation may need to be invoked to control excessive immune reactions. Augmenting EMR2 signaling through TGFβ may be protective because it will reduce inflammation and control damage to the surrounding tissues.

Using gene therapy, one can manipulate the EMR2 gene construct and subsequent expression in an area of interest in order to induce or target immune privilege. It has been shown extensively by the Streilein and Stein-Streilein groups that in vitro generated tolerogenic APCs are able to retain their functionality when reintroduced into a living organism. Generating tolerogenic cells in vitro and re-educating immune cells to become regulatory before re-injecting them into the area of interest may be a near future method to target localized inflammation. TGFβ treatment into a local tissue may also have the same effect. Thus knowing that tolerogenic APCs express EMR2 may be a basis for future therapies regarding immune and inflammation-related diseases.

To test the efficacy of blocking EMR2 via monoclonal antibody specific to the stalk region of EMR2, the following experiments were conducted. While soluble chondroitin sulfate successfully blocks Treg production, it may not be binding specifically to EMR2 only. Armenian hamster anti-human CD97/EMR2 EGF domain 4 monoclonal antibody (clone 1b5) binds to the stalk region of EMR2. 1b5 will abrogates EMR2 signaling and abolish Treg populations or significantly decrease Treg populations. Activity is tested by measuring CD4+CD25+ FoxP3+ Treg populations (and their intracellular cytokine production) after APC co-culture with or without 1b5. Tregs are then tested in a humanized NOD SCID mouse model and subjected to a DTH response. The suppression or lack of suppression of DTH is used as a functional readout of Treg function.

Treg Cell Generation with and without 1b5

Day zero, APC were isolated from whole huPBMC as stated before and plated in serum-free medium with or without TGFb and Tetanus toxoid (TT) antigen. In the experimental condition, we added 5 ug/ml 1b5 and allowed all dishes to incubate overnight. On Day one, APC were washed two times and autologous CD3+ lymphocytes were added at a ratio of 2.5:1. Cells were incubated for five days and analyzed for CD4 and CD25 surface markers; FoxP3, IL-10, and IFN-g intracellular cytokines.

In Vivo Test of Treg Suppression of DTH with and without 1b5

NOD SCID mice were humanized by injecting huPBMC intraperitoneally on Day zero. On Day one, mice were injected subcutaneously with TT antigen (except for negative control animals). On Day eight, mice were subjected to a localized ear challenge with or without Tregs (generated as above). On Day nine, mice ear thickness was measured as a functional readout of DTH response.

A decrease in the expression of FoxP3 (on CD4+CD25+ T cells) with the addition of 1b5 was observed (FIG. 13B). A significant abrogation in IL-10 production of cells in co-culture with 1b5-treated APCs as well as an increase in IFNg production was observed (FIG. 13C).

With APCs treated with 1b5, the decreased Treg populations indicate that the mAb successfully blocked EMR2 signaling with more specificity than soluble chondroitin sulfate. By targeting a specified region of the EMR2 molecule, the specific antibody blocking indicated that EMR2 is required in the significant generation of Treg cells to a foreign antigen, TT.

By blocking EMR2, the APCs can no longer upregulate Treg populations, thus they remain with a pro-inflammatory cytokine profile. Thus, with this mAb, Treg populations against a specific antigen can be manipulated depending on the need of a certain disease state.

EMR2 is an adhesion G protein-coupled receptor (ad-GPCR) belonging to a subfamily called epidermal growth factor-seven transmembrane receptors (EGF-TM7). All members of this family possess a large extracellular region often containing common protein modules, such as immunoglobulin, epidermal growth factor-like (EGF), lectin, or thrombospondin repeats, coupled to a seven-transmembrane (TM7) domain via a mucin-like stalk region. EMR2 is expressed on neutrophils, monocytes, macrophages, and dendritic cells.

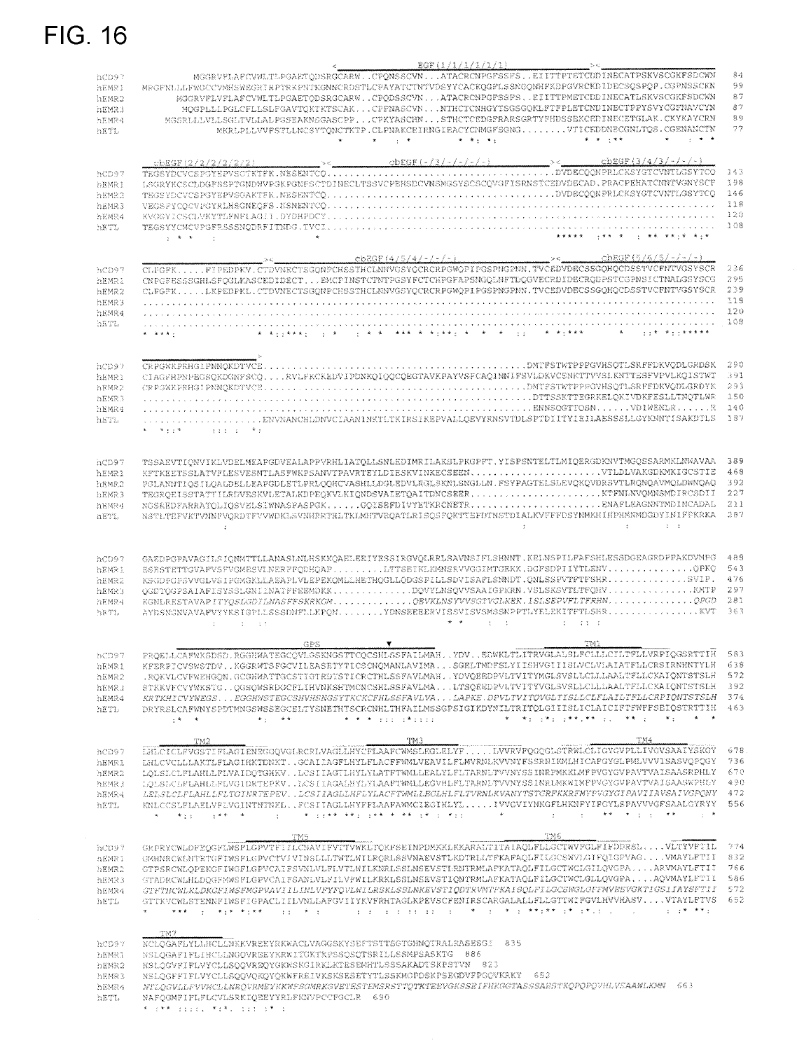

A ligand of EMR2 is chondroitin sulfate (structure shown below). Chondroitin sulfate is a glycosaminoglycan side chain abundantly found as component of cell surfaces proteoglycans and in extracellular matrixes. Binding to chondroitin sulfate is mediated by the fourth EGF domain of EMR2. The residue Arg241 of EMR2 has been identified to be important for such binding (see human EMR2 sequence in FIG. 16).

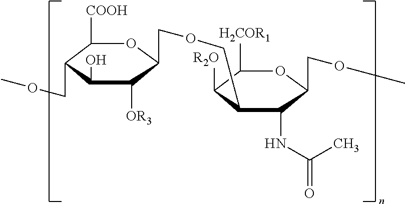

EMR2 amino acid sequence is shown below.

```
Q9UHX3|EMR2_HUMAN EGF-like module-containing mucin-like hormone receptor-
like 2 OS = Homo sapiens GN = EMR2 PE = 1 SV = 2 (Gene ID: 30817)
(SEQ ID NO: 1)
MGGRVFLVFLAFCVWLTLPGAETQDSRGCARWCPQDSSCVNATACRCNPGFSSFSEIITTPMETCDDINE
CATLSKVSCGKFSDCWNTEGSYDCVCSPGYEPVSGAKTFKNESENTCQDVDECQQNPRLCKSYGTCVNTL
GSYTCQCLPGFKLKPEDPKLCTDVNECTSGQNPCHSSTHCLNNVGSYQCRCRPGWQPIPGSPNGPNNTVC
EDVDECSSGQHQCDSSTVCFNTVGSYSCRCRPGWKPRHGIPNNQKDTVCEDMTFSTWTPPPGVHSQTLSR
FFDKVQDLGRDYKPGLANNTIQSILQALDELLEAPGDLETLPRLQQHCVASHLLDGLEDVLRGLSKNLSN
GLLNFSYPAGTELSLEVQKQVDRSVTLRQNQAVMQLDWNQAQKSGDPGPSVVGLVSIPGMGKLLAEAPLV
LEPEKQMLLHETHQGLLQDGSPILLSDVISAFLSNNDTQNLSSPVTFTFSHRSVIPRQKVLCVFWEHGQN
GCGHWATTGCSTIGTRDTSTICRCTHLSSFAVLMAHYDVQEEDPVLTVITYMGLSVSLLCLLLAALTFLL
CKAIQNTSTSLHLQLSLCLFLAHLLFLVAIDQTGHKVLCSIIAGTLHYLYLATLTWMLLEALYLFLTARN
LTVVNYSSINRFMKKLMFPVGYGVPAVTVAISAASRPHLYGTPSRCWLQPEKGFIWGFLGPVCAIFSVNL
VLFLVTLWILKNRLSSLNSEVSTLRNTRMLAFKATAQLFILGCTWCLGILQVGPAARVMAYLFTIINSLQ
GVFIFLVYCLLSQQVREQYGKWSKGIRKLKTESEMHTLSSSAKADTSKPSTVN
(underlined is present in epitope binding region of EMR2 specific
monoclonal antibody)
(SEQ ID NO: 2)
DECSSGQHQCDSSTVCFNTVGSYSCRCRPGWKPRHGIPNNQKDTVCEDMTFSTWTPPPGVHSQTLSRFFD
KVQDLGRDYKPGLANNTIQSILQALDELLEAPGDLETLPRLQQHCVASHLLDGLEDVLRGLSKNLSNGLL
NFSYPAGTELSLEVQKQVDRSVTLRQNQAVMQLDWNQAQKSGDPGPSVVGLVSIPGMGKLLAEAPLVLEP
EKQMLLHETHQGLLQDGSPILLSDVISAFLSNNDTQNLSSPVTFTFSHRSVIPRQKV
Q9UHX3-2|EMR2_HUMAN Isoform 2 of EGF-like module-containing mucin-like
hormone receptor-like 2 OS = Homo sapiens GN = EMR2
(SEQ ID NO: 3)
MGGRVFLVFLAFCVWLTLPGAETQDSRGCARWCPQDSSCVNATACRCNPGFSSFSEIITTPMETCDDINE
CATLSKVSCGKFSDCWNTEGSYDCVCSPGYEPVSGAKTFKNESENTCQDVDECQQNPRLCKSYGTCVNTL
GSYTCQCLPGFKLKPEDPKLCTDVNECTSGQNPCHSSTHCLNNVGSYQCRCRPGWQPIPGSPNGPNNTVC
EDVDECSSGQHQCDSSTVCFNTVGSYSCRCRPGWKPRHGIPNNQKDTVCEDMTFSTWTPPPGVHSQTLSR
FFDKVQDLGRDYKPGLANNTIQSILQALDELLEAPGDLETLPRLQQHCVASHLLDGLEDVLRGLSKNLSN
GLLNFSYPAGTELSLEVQKQVDRSVTLRQNQAVMQLDWNQAQKSGDPGMGKLLAEAPLVLEPEKQMLLHE
THQGLLQDGSPILLSDVISAFLSNNDTQNLSSPVTFTFSHRSVIPRQKVLCVFWEHGQNGCGHWATTGCS
TIGTRDTSTICRCTHLSSFAVLMAHYDVQEEDPVLTVITYMGLSVSLLCLLLAALTFLLCKAIQNTSTSL
HLQLSLCLFLAHLLFLVAIDQTGHKVLCSIIAGTLHYLYLATLTWMLLEALYLFLTARNLTVVNYSSINR
FMKKLMFPVGYGVPAVTVAISAASRPHLYGTPSRCWLQPEKGFIWGFLGPVCAIFSVNLVLFLVTLWILK
NRLSSLNSEVSTLRNTRMLAFKATAQLFILGCTWCLGILQVGPAARVMAYLFTIINSLQGVFIFLVYCLL
SQQVREQYGKWSKGIRKLKTESEMHTLSSSAKADTSKPSTVN
Q9UHX3-3|EMR2_HUMAN Isoform 3 of EGF-like module-containing mucin-like
hormone receptor-like 2 OS = Homo sapiens GN = EMR2
(SEQ ID NO: 4)
MGGRVFLVFLAFCVWLTLPGAETQDSRGCARWCPQDSSCVNATACRCNPGFSSFSEIITTPMETCDDINE
CATLSKVSCGKFSDCWNTEGSYDCVCSPGYEPVSGAKTFKNESENTCQDVDECQQNPRLCKSYGTCVNTL
GSYTCQCLPGFKLKPEDPKLCTDVDECSSGQHQCDSSTVCFNTVGSYSCRCRPGWKPRHGIPNNQKDTVC
EDMTFSTWTPPPGVHSQTLSRFFDKVQDLGRDYKPGLANNTIQSILQALDELLEAPGDLETLPRLQQHCV
ASHLLDGLEDVLRGLSKNLSNGLLNFSYPAGTELSLEVQKQVDRSVTLRQNQAVMQLDWNQAQKSGDPGP
SVVGLVSIPGMGKLLAEAPLVLEPEKQMLLHETHQGLLQDGSPILLSDVISAFLSNNDTQNLSSPVTFTF
SHRSVIPRQKVLCVFWEHGQNGCGHWATTGCSTIGTRDTSTICRCTHLSSFAVLMAHYDVQEEDPVLTVI
TYMGLSVSLLCLLLAALTFLLCKAIQNTSTSLHLQLSLCLFLAHLLFLVAIDQTGHKVLCSIIAGTLHYL
YLATLTWMLLEALYLFLTARNLTVVNYSSINRFMKKLMFPVGYGVPAVTVAISAASRPHLYGTPSRCWLQ
PEKGFIWGFLGPVCAIFSVNLVLFLVTLWILKNRLSSLNSEVSTLRNTRMLAFKATAQLFILGCTWCLGI
LQVGPAARVMAYLFTIINSLQGVFIFLVYCLLSQQVREQYGKWSKGIRKLKTESEMHTLSSSAKADTSKP
STVN
Q9UHX3-4_EMR2_HUMAN Isoform 4 of EGF-like module-containing mucin-like
hormone receptor-like 2 OS = Homo sapiens GN = EMR2
(SEQ ID NO: 5)
MGGRVFLVFLAFCVWLTLPGAETQDSRGCARWCPQDSSCVNATACRCNPGFSSFSEIITTPMETCDDINE
CATLSKVSCGKFSDCWNTEGSYDCVCSPGYEPVSGAKTFKNESENTCQDVDECSSGQHQCDSSTVCFNTV
GSYSCRCRPGWKPRHGIPNNQKDTVCEDMTFSTWTPPPGVHSQTLSRFFDKVQDLGRDYKPGLANNTIQS
ILQALDELLEAPGDLETLPRLQQHCVASHLLDGLEDVLRGLSKNLSNGLLNFSYPAGTELSLEVQKQVDR
SVTLRQNQAVMQLDWNQAQKSGDPGPSVVGLVSIPGMGKLLAEAPLVLEPEKQMLLHETHQGLLQDGSPI
LLSDVISAFLSNNDTQNLSSPVTFTFSHRSVIPRQKVLCVFWEHGQNGCGHWATTGCSTIGTRDTSTICR
CTHLSSFAVLMAHYDVQEEDPVLTVITYMGLSVSLLCLLLAALTFLLCKAIQNTSTSLHLQLSLCLFLAH
LLFLVAIDQTGHKVLCSIIAGTLHYLYLATLTWMLLEALYLFLTARNLTVVNYSSINRFMKKLMFPVGYG
VPAVTVAISAASRPHLYGTPSRCWLQPEKGFIWGFLGPVCAIFSVNLVLFLVTLWILKNRLSSLNSEVST
LRNTRMLAFKATAQLFILGCTWCLGILQVGPAARVMAYLFTIINSLQGVFIFLVYCLLSQQVREQYGKWS
KGIRKLKTESEMHTLSSSAKADTSKPSTVN
Q9UHX3-5|EMR2_HUMAN Isoform 5 of EGF-like module-containing mucin-like
hormone receptor-like 2 OS = Homo sapiens GN = EMR2
(SEQ ID NO: 6)
MGGRVFLVFLAFCVWLTLPGAETQDSRGCARWCPQDSSCVNATACRCNPGFSSFSEIITTPMETCDDINE
CATLSKVSCGKFSDCWNTEGSYDCVCSPGYEPVSGAKTFKNESENTCQDMTFSTWTPPPGVHSQTLSRFF
DKVQDLGRDYKPGLANNTIQSILQALDELLEAPGDLETLPRLQQHCVASHLLDGLEDVLRGLSKNLSNGL
LNFSYPAGTELSLEVQKQVDRSVTLRQNQAVMQLDWNQAQKSGDPGPSVVGLVSIPGMGKLLAEAPLVLE
PEKQMLLHETHQGLLQDGSPILLSDVISAFLSNNDTQNLSSPVTFTFSHRSVIPRQKVLCVFWEHGQNGC
GHWATTGCSTIGTRDTSTICRCTHLSSFAVLMAHYDVQEEDPVLTVITYMGLSVSLLCLLLAALTFLLCK
AIQNTSTSLHLQLSLCLFLAHLLFLVAIDQTGHKVLCSIIAGTLHYLYLATLTWMLLEALYLFLTARNLT
VVNYSSINRFMKKLMFPVGYGVPAVTVAISAASRPHLYGTPSRCWLQPEKGFIWGFLGPVCAIFSVNLVL
FLVTLWILKNRLSSLNSEVSTLRNTRMLAFKATAQLFILGCTWCLGILQVGPAARVMAYLFTIINSLQGV
FIFLVYCLLSQQVREQYGKWSKGIRKLKTESEMHTLSSSAKADTSKPSTVN
Q9UHX3-6IEMR2 HUMAN Isoform 6 of EGF-like module-containing mucin-like
hormone receptor-like 2 OS = Homo sapiens GN = EMR2
(SEQ ID NO: 7)
MGGRVFLVFLAFCVWLTLPGAETQDSRGCARWCPQDSSCVNATACRCNPGFSSFSEIITTPMETCDDINE
CATLSKVSCGKFSDCWNTEGSYDCVCSPGYEPVSGAKTFKNESENTCQDVDECQQNPRLCKSYGTCVNTL
GSYTCQCLPGFKLKPEDPKLCTDVNECTSGQNPCHSSTHCLNNVGSYQCRCRPGWQPIPGSPNGPNNTVC
EDVDECSSGQHQCDSSTVCFNTVGSYSCRCRPGWKPRHGIPNNQKDTVCEDMTFSTWTPPPGVHSQTLSR
FFDKVQDLGRDYKPGLANNTIQSILQALDELLEAPGDLETLPRLQQHCVASHLLDGLEDVLRGLSKNLSN
GLLNFSYPAGTELSLEVQKQVDRSVTLRQNQAVMQLDWNQAQKSGDPGPSVVGLVSIPGMGKLLAEAPLV
LEPEKQMLLHETHQGLLQDGSPILLSDVISAFLSNNDTQNLSSPVTFTFSHREEDPVLTVITYMGLSVSL
LCLLLAALTFLLCKAIQNTSTSLHLQLSLCLFLAHLLFLVAIDQTGHKVLCSIIAGTLHYLYLATLTWML
LEALYLFLTARNLTVVNYSSINRFMKKLMFPVGYGVPAVTVAISAASRPHLYGTPSRCWLQPEKGFIWGF
LGPVCAIFSVNLVLFLVTLWILKNRLSSLNSEVSTLRNTRMLAFKATAQLFILGCTWCLGILQVGPAARV
MAYLFTIINSLQGVFIFLVYCLLSQQVREQYGKWSKGIRKLKTESEMHTLSSSAKADTSKPSTVN
Homo sapiens egf-like module containing, mucin-like, hormone receptor-like
2 (EMR2), transcript variant 8, mRNA
NCBI Reference Sequence: NM_001271052.1
VERSION NM_001271052.1 GI: 402743848
(SEQ ID NO: 8)
      1 ctgcaccgcc agttccgggg agggccctgg gccagcggct gtccgccccc cctcctttat
    61 aaagtcctgg cctcgggaca gcccgcacag ctgcccagcc tgcggagacg ggacagccct
  121 gtcccactca ctctttcccc tgctgctcct gccggcagct cagctggaac catgggaggc
  181 cgcgtctttc tcgtctttct cgcattctgt gtctggctga ctctgccggg agctgaaacc
  241 caggactcca ggggctgtgc ccggtggtgc cctcaggact cctcgtgtgt caatgccacc
  301 gcctgtcgct gcaatccagg gttcagctct ttttctgaga tcatcaccac ccccatggag
  361 acttgtgacg acatcaacga gtgtgcaaca ctgtcgaaag tgtcatgcgg aaaattctcg
  421 gactgctgga acacagaggg gagctacgac tgcgtgtgca gcccaggata tgagcctgtt
  481 tctggggcaa aaacattcaa gaatgagagc gagaacacgt gtcaagatgt ggacgaatgt
  541 cagcagaacc caaggctctg taaaagctac ggcacctgcg tcaacaccct cggcagctac
  601 acgtgccagt gcctgcctgg cttcaagctc aaacctgagg acccgaagct ctgcacagat
  661 gtgaatgaat gcacctccgg acaaaaccca tgccacagct ccacccactg cctcaacaac
  721 gtgggcagct atcagtgccg ctgccgcccg ggctggcaac cgattccggg gtcccccaat
  781 ggcccaaaca ataccgtctg tgaagatgtg gacgagtgca gctccgggca gcatcagtgt
  841 gacagctcca ccgtctgctt caacaccgtg ggttcataca gctgccgctg ccgcccaggc
  901 tggaagccca gacacggaat cccgaataac caaaaggaca ctgtctgtga agatatgact
  961 ttctccacct ggaccccgcc ccctggagtc cacagccaga cgctttcccg attcttcgac
1021 aaagtccagg acctgggcag agactacaag ccaggcttgg ccaataacac catccagagc
1081 atcttacagg cgctggatga gctgctggag gcccctgggg acctggagac cctgccccgc
1141 ttacagcagc actgtgtggc cagtcacctg ctggatggcc tagaggatgt cctcagaggc
1201 ctgagcaaga acctttccaa tgggctgttg aacttcagtt atcctgcagg cacagaattg
1261 tccctggagg tgcagaagca agtagacagg agtgtcacct tgagacagaa tcaggcagtg
1321 atgcagctcg actggaatca ggcacagaaa tctggtgacc caggcccttc tgtggtgggc
1381 cttgtctcca ttccagggat gggcaagttg ctggctgagg cccctctggt cctggaacct
1441 gagaagcaga tgcttctgca tgagacacac cagggcttgc tgcaggacgg ctcccccatc
1501 ctgctctcag atgtgatctc tgcctttctg agcaacaacg acacccaaaa cctcagctcc
1561 ccagttacct tcaccttctc ccaccgtgag gaggatcccg tgctgactgt catcacctac
1621 atggggctga gcgtctctct gctgtgcctc ctcctggcgg ccctcacttt tctcctgtgt
1681 aaagccatcc agaacaccag cacctcactg catctgcagc tctcgctctg cctcttcctg
1741 gcccacctcc tcttcctcgt ggcaattgat caaaccggac acaaggtgct gtgctccatc
1801 atcgccggta ccttgcacta tctctacctg gccaccttga cctggatgct gctggaggcc
1861 ctgtacctct tcctcactgc acggaacctg acggtggtca actactcaag catcaacaga
1921 ttcatgaaga agctcatgtt ccctgtgggc tacggagtcc cagctgtgac agtggccatt
1981 tctgcagcct ccaggcctca cctttatgga acaccttccc gctgctggct ccaaccagaa
2041 aagggattta tatggggctt ccttggacct gtctgcgcca tcttctctgt gaatttagtt
2101 ctctttctgg tgactctctg gattttgaaa aacagactct cctccctcaa tagtgaagtg
2161 tccaccctcc ggaacacaag gatgctggca tttaaagcga cagctcagct gttcatcctg
2221 ggctgcacgt ggtgtctggg catcttgcag gtgggtccgg ctgcccgggt catggcctac
2281 ctcttcacca tcatcaacag cctgcagggt gtcttcatct tcctggtgta ctgcctcctc
2341 agccagcagg tccgggagca atatgggaaa tggtccaaag ggatcaggaa attgaaaact
2401 gagtctgaga tgcacacact ctccagcagt gctaaggctg acacctccaa acccagcacg
2461 gttaactaga aaaatcttct gaataagatc ttccctcttt gcccgtggaa aatctgaaca
2521 atctttgagc catctagagg ggaaagaaaa gactttgttc tgtgtgtttc aagaaattca
2581 ccatgtcagc aatatgaagg atgttatgga aggcgtgcta ggcattcaat tcctgcagaa
2641 accggaaatc ttccatgccc tgcaatgtgc tcatcaaact ctcagcatat ggacggccag
2701 ctgtggccca tatcttggtc actctgaagc acaatattta tgaagctata gaacgttaag
2761 acctctttca cagcctctcc ttcctacaaa gactcctcca aatcttaaaa tgaagcagga
2821 aaacgagcct aagaggactt tcataccgac aacatctgaa aggactagaa tgttcacacc
2881 acgatctgga tttcttaatt ttttgttttt gtttttgttg ttctctagtt ctacgggttt
2941 gattatttag tcatgtgaaa aatattgatt actcacacat agatcaagag agacacggct
3001 cctgccttca tggagctttt aggggaaaat gaagtggctc ttgcagctag agttgactca
3061 gaagccgaaa ttcctagaaa tcaggtttct actgctaggc aattgaagta taaactattt
3121 tataaacact gtcttctttc gtcttcacac caacatgcag aaaagtttct aatctcagat
3181 cggggatgtg caacaaattc catttcaaag gaatgacctg caaaactcct aaatattcca
3241 agcaaatgcc cttaaccctg tctgttatct gctttccttg aacagaaatt ctacatgacc
3301 ataaaacctc gaagatgggt atggcacagt tcatgccctg taatcctagc actttgggag
3361 ggtgaggcag gaggatggct caagcccagg agtttgagac cagtgtgggc aacagagtga
3421 gaaccatctc tacccaaaaa aaaaattaaa aattagccaa gcatggtgat gatataggag
3481 ttaaggagaa atcatttagg caaatagcaa gggtaggaag tcctcagtaa ggttttccat
3541 ttaatgaaaa gcagccccca aaatcatttt cttttctaac aaagaacagc ctgtaaaatc
3601 gagctgcaga catagacaag caagctggaa gcttccacgg gtgaatgccg gcagctgtgc
3661 caataggaaa aagctaccta gactaggcat gtccaaaatg gcggctccaa gttcccttct
3721 ctttgccagc catgtgtaca gtaaaaagca ggcaacatag tgtcagccaa agctcatttg
3781 cataataaga ttagggtggg gtggccagct cacatagggg taggccctag gtaaatcaga
3841 caccgccttc tcaagcctgt ctataaaatc tggtacacta tgacgagggt cagatttccc
3901 attcagacgc ccctctccca tgcaagagaa agagctgttc tcctttctct ttcttttgcc
3961 tattaaacct ctgctcctgg ccaggcacag tggctcacgc ctataatccc agcactttgg
4021 gaggctgagg tggtcagatc acctaaggtc aggagttcaa gaccagcctg gtcaacatgg
4081 tgaaatcttg tctctagtaa aaatacaaaa atatatgaaa tctcacatag atgataatat
4141 taagttccaa aagcaactca acctggtaga ttctaatttt ttttgaggca gggtcttgct
4201 ttgtcaccca tgctggagta caatggcaca aacactgctc actgcagcct cgacctccca
4261 aggcctaagc aatcctcctg cctcagtccc cctccaggta tttgaaacta caggtgtgta
4321 ccaccacacc gggctaattt ttgtattttt tgtagagacg tgggtctcac tatgctgccc
4381 aggctcaggt cttaatctcc tgagctcagg caatccgcag gcctcagcct ccctaagtgc
4441 ggggattaca ggcttgagcc actgcaccta gcctctattt gttttacaaa agagaaattg
4501 agatcctgaa tgttaagtga cttgcctgag gccatcccac taacaggagc cagggttagg
4561 attcaaaccc catccaactg gtcccagagc tggagcttct tgcactgccc tacactacct
4621 accatctcca tcctctgggc acctttttat aagaaccaaa acattacaga gcattgcttt
4681 gtcaactcag ctgggaacat ttcccagtgc aactcacatt tttcactgct ctgtgcctgt
4741 ccgtataagc tcaatgagta ttgatttagg ggctttggag aactttgaat gctacccccc
4801 aagtaaccat tgttggcaac ctggtacctc tacttttagc catttctcct tctctataaa
4861 tagtgcagaa gtaacccact tggtaacagg catccttgcc aagcctccac cactaggtca
4921 gtgtaagaat taaagaaaga ggaaagaaac acaaaaagtg gcttgatggt taagacaggt
4981 ttattttaga gaaaacacac ctgagagggg ctgctggctg aattaggtta gagtcttttc
5041 tacagactaa gagtgtttaa ggatttaggg tgggagagtt tcttagaggc ttggactgct
5101 tctgtgtttt ttttgttgtg cttatatggg agggagagtg gtgtgtttgc ttttatacat
5161 ttttctgcag ctgtaggcat accccccaag tctgctttta gcttccctat tttagtgcac
5221 ctggagggaa aggaatgtgc ttattaaggc ccactgtttt actggggccc attgtatgag
5281 ggtgaagttt ggcagttacc caagagactt ttcctccacc ttcctctgtg cccgagctgt
5341 tttatctgca ttttactgtc tgcttttttt ggctgcttat agtttttaaa aaagtaattt
5401 ccttaaatcc agaaggctaa aaatgaagct gaaacttaaa gtggcggtgt ttgtccaaaa
5461 taacggggct cctgctctgc cagtcagtac cctcaagtca ctcctgatcc tcaacctcca
5521 tgcctaaggc tggttcaaga gaccacataa tatctgcctt ttattacata catgatgggt
5581 gcatgggatt ctgcgtgccc tttgcttgat atagactgct aaggtgagat ggggaatatc
5641 agagtcagct gctgcttgag gaagcagaac acacagctgg aggcttggaa catgtgggtc
5701 cctatgagtg tagagcccat atccccatag agtctaccta gagcaggggt cgccaaatgt
5761 tttcttaaag agcctgatag tgtatatgtt aggctttgtg agccaggtat ttacagcaac
5821 tcaattctac cactgtggta tgaaaacagc tatagacaat cataaatgaa tgatcatggc
5881 tatgttttaa taaaacttta cagacactga acttgaactt ccattgtgat atgaaaacag
5941 ctatagacaa tcataaatga atgatcatgg ctatgtttta ataaaacttt atggacactg
6001 agcttgaaaa aaaaaaaaaa aaaa
Translation
(SEQ ID NO: 9)
MGGRVFLVFLAFCVWLTLPGAETQDSRGCARWCPQDSSCVNATACRCNPGFSSFSEIITTPMETCDDINE
CATLSKVSCGKFSDCWNTEGSYDCVCSPGYEPVSGAKTFKNESENTCQDVDECQQNPRLCKSYGTCVNTL
GSYTCQCLPGFKLKPEDPKLCTDVNECTSGQNPCHSSTHCLNNVGSYQCRCRPGWQPIPGSPNGPNNTVC
EDVDECSSGQHQCDSSTVCFNTVGSYSCRCRPGWKPRHGIPNNQKDTVCEDMTFSTWTPPPGVHSQTLSR
FFDKVQDLGRDYKPGLANNTIQSILQALDELLEAPGDLETLPRLQQHCVASHLLDGLEDVLRGLSKNLSN
GLLNFSYPAGTELSLEVQKQVDRSVTLRQNQAVMQLDWNQAQKSGDPGPSVVGLVSIPGMGKLLAEAPLV
LEPEKQMLLHETHQGLLQDGSPILLSDVISAFLSNNDTQNLSSPVTFTFSHREEDPVLTVITYMGLSVSL
LCLLLAALTFLLCKAIQNTSTSLHLQLSLCLFLAHLLFLVAIDQTGHKVLCSIIAGTLHYLYLATLTWML
LEALYLFLTARNLTVVNYSSINRFMKKLMFPVGYGVPAVTVAISAASRPHLYGTPSRCWLQPEKGFIWGF
LGPVCAIFSVNLVLFLVTLWILKNRLSSLNSEVSTLRNTRMLAFKATAQLFILGCTWCLGILQVGPAARV
MAYLFTIINSLQGVFIFLVYCLLSQQVREQYGKWSKGIRKLKTESEMHTLSSSAKADTSKPSTVN
Homo sapiens egf-like module containing, mucin-like, hormone receptor-like
2 (EMR2), transcript variant 1, mRNA
NCBI Reference Sequence: NM_013447.3
VERSION NM_013447.3 GI: 402743834
(SEQ ID NO: 10)
      1 ggggggtctg ctctgtgcgg tgaagcttct cttcttggca cctgcctggc atcggaagag
    61 ggccccttct ccctccctgg gcttttatgt ggacactgta atgcctcagt tttctttctt
  121 tctttttgtt tttgacacag ggtctcactc tatcacccaa gctggagtgc agtggcacca
  181 tcttagctca ccgcagcctc aaactcccca gctcgggtga ttcttcctgc ctcagcctcc
  241 tgagtagtag cagctgggac cacagacgtc tgccaccaag cccagctgca ccgccagttc
  301 cggggagggc cctgggccag cggctgtccg ccccccctcc tttataaagt cctggcctcg
  361 ggacagcccg cacagctgcc cagcctgcgg agacgggaca gccctgtccc actcactctt
  421 tcccctgctg ctcctgccgg cagctcagct ggaaccatgg gaggccgcgt ctttctcgtc
  481 tttctcgcat tctgtgtctg gctgactctg ccgggagctg aaacccagga ctccaggggc
  541 tgtgcccggt ggtgccctca ggactcctcg tgtgtcaatg ccaccgcctg tcgctgcaat
  601 ccagggttca gctctttttc tgagatcatc accaccccca tggagacttg tgacgacatc
  661 aacgagtgtg caacactgtc gaaagtgtca tgcggaaaat tctcggactg ctggaacaca
  721 gaggggagct acgactgcgt gtgcagccca ggatatgagc ctgtttctgg ggcaaaaaca
  781 ttcaagaatg agagcgagaa cacgtgtcaa gatgtggacg aatgtcagca gaacccaagg
  841 ctctgtaaaa gctacggcac ctgcgtcaac accctcggca gctacacgtg ccagtgcctg
  901 cctggcttca agctcaaacc tgaggacccg aagctctgca cagatgtgaa tgaatgcacc
  961 tccggacaaa acccatgcca cagctccacc cactgcctca acaacgtggg cagctatcag
1021 tgccgctgcc gcccgggctg gcaaccgatt ccggggtccc ccaatggccc aaacaatacc
1081 gtctgtgaag atgtggacga gtgcagctcc gggcagcatc agtgtgacag ctccaccgtc
1141 tgcttcaaca ccgtgggttc atacagctgc cgctgccgcc caggctggaa gcccagacac
1201 ggaatcccga ataaccaaaa ggacactgtc tgtgaagata tgactttctc cacctggacc
1261 ccgccccctg gagtccacag ccagacgctt tcccgattct tcgacaaagt ccaggacctg
1321 ggcagagact acaagccagg cttggccaat aacaccatcc agagcatctt acaggcgctg
1381 gatgagctgc tggaggcccc tggggacctg gagaccctgc cccgcttaca gcagcactgt
1441 gtggccagtc acctgctgga tggcctagag gatgtcctca gaggcctgag caagaacctt
1501 tccaatgggc tgttgaactt cagttatcct gcaggcacag aattgtccct ggaggtgcag
1561 aagcaagtag acaggagtgt caccttgaga cagaatcagg cagtgatgca gctcgactgg
1621 aatcaggcac agaaatctgg tgacccaggc ccttctgtgg tgggccttgt ctccattcca
1681 gggatgggca agttgctggc tgaggcccct ctggtcctgg aacctgagaa gcagatgctt
1741 ctgcatgaga cacaccaggg cttgctgcag gacggctccc ccatcctgct ctcagatgtg
1801 atctctgcct ttctgagcaa caacgacacc caaaacctca gctccccagt taccttcacc
1861 ttctcccacc gttcagtgat cccgagacag aaggtgctct gtgtcttctg ggagcatggc
1921 cagaatggat gtggtcactg ggccaccaca ggctgcagca caataggcac cagagacacc
1981 agcaccatct gccgttgcac ccacctgagc agctttgccg tcctcatggc ccactacgat
2041 gtgcaggagg aggatcccgt gctgactgtc atcacctaca tggggctgag cgtctctctg
2101 ctgtgcctcc tcctggcggc cctcactttt ctcctgtgta aagccatcca gaacaccagc
2161 acctcactgc atctgcagct ctcgctctgc ctcttcctgg cccacctcct cttcctcgtg
2221 gcaattgatc aaaccggaca caaggtgctg tgctccatca tcgccggtac cttgcactat
2281 ctctacctgg ccaccttgac ctggatgctg ctggaggccc tgtacctctt cctcactgca
2341 cggaacctga cggtggtcaa ctactcaagc atcaacagat tcatgaagaa gctcatgttc
2401 cctgtgggct acggagtccc agctgtgaca gtggccattt ctgcagcctc caggcctcac
2461 ctttatggaa caccttcccg ctgctggctc caaccagaaa agggatttat atggggcttc
2521 cttggacctg tctgcgccat cttctctgtg aatttagttc tctttctggt gactctctgg
2581 attttgaaaa acagactctc ctccctcaat agtgaagtgt ccaccctccg gaacacaagg
2641 atgctggcat ttaaagcgac agctcagctg ttcatcctgg gctgcacgtg gtgtctgggc
2701 atcttgcagg tgggtccggc tgcccgggtc atggcctacc tcttcaccat catcaacagc
2761 ctgcagggtg tcttcatctt cctggtgtac tgcctcctca gccagcaggt ccgggagcaa
2821 tatgggaaat ggtccaaagg gatcaggaaa ttgaaaactg agtctgagat gcacacactc
2881 tccagcagtg ctaaggctga cacctccaaa cccagcacgg ttaactagaa aaatcttctg
2941 aataagatct tccctctttg cccgtggaaa atctgaacaa tctttgagcc atctagaggg
3001 gaaagaaaag actttgttct gtgtgtttca agaaattcac catgtcagca atatgaagga
3061 tgttatggaa ggcgtgctag gcattcaatt cctgcagaaa ccggaaatct tccatgccct
3121 gcaatgtgct catcaaactc tcagcatatg gacggccagc tgtggcccat atcttggtca
3181 ctctgaagca caatatttat gaagctatag aacgttaaga cctctttcac agcctctcct
3241 tcctacaaag actcctccaa atcttaaaat gaagcaggaa aacgagccta agaggacttt
3301 cataccgaca acatctgaaa ggactagaat gttcacacca cgatctggat ttcttaattt
3361 tttgtttttg tttttgttgt tctctagttc tacgggtttg attatttagt catgtgaaaa
3421 atattgatta ctcacacata gatcaagaga gacacggctc ctgccttcat ggagctttta
3481 ggggaaaatg aagtggctct tgcagctaga gttgactcag aagccgaaat tcctagaaat
3541 caggtttcta ctgctaggca attgaagtat aaactatttt ataaacactg tcttctttcg
3601 tcttcacacc aacatgcaga aaagtttcta atctcagatc ggggatgtgc aacaaattcc
3661 atttcaaagg aatgacctgc aaaactccta aatattccaa gcaaatgccc ttaaccctgt
3721 ctgttatctg ctttccttga acagaaattc tacatgacca taaaacctcg aagatgggta
3781 tggcacagtt catgccctgt aatcctagca ctttgggagg gtgaggcagg aggatggctc
3841 aagcccagga gtttgagacc agtgtgggca acagagtgag aaccatctct acccaaaaaa
3901 aaaattaaaa attagccaag catggtgatg atataggagt taaggagaaa tcatttaggc
3961 aaatagcaag ggtaggaagt cctcagtaag gttttccatt taatgaaaag cagcccccaa
4021 aatcattttc ttttctaaca aagaacagcc tgtaaaatcg agctgcagac atagacaagc
4081 aagctggaag cttccacggg tgaatgccgg cagctgtgcc aataggaaaa agctacctag
4141 actaggcatg tccaaaatgg cggctccaag ttcccttctc tttgccagcc atgtgtacag
4201 taaaaagcag gcaacatagt gtcagccaaa gctcatttgc ataataagat tagggtgggg
4261 tggccagctc acataggggt aggccctagg taaatcagac accgccttct caagcctgtc
4321 tataaaatct ggtacactat gacgagggtc agatttccca ttcagacgcc cctctcccat
4381 gcaagagaaa gagctgttct cctttctctt tcttttgcct attaaacctc tgctcctggc
4441 caggcacagt ggctcacgcc tataatccca gcactttggg aggctgaggt ggtcagatca
4501 cctaaggtca ggagttcaag accagcctgg tcaacatggt gaaatcttgt ctctagtaaa
4561 aatacaaaaa tatatgaaat ctcacataga tgataatatt aagttccaaa agcaactcaa
4621 cctggtagat tctaattttt tttgaggcag ggtcttgctt tgtcacccat gctggagtac
4681 aatggcacaa acactgctca ctgcagcctc gacctcccaa ggcctaagca atcctcctgc
4741 ctcagtcccc ctccaggtat ttgaaactac aggtgtgtac caccacaccg ggctaatttt
4801 tgtatttttt gtagagacgt gggtctcact atgctgccca ggctcaggtc ttaatctcct
4861 gagctcaggc aatccgcagg cctcagcctc cctaagtgcg gggattacag gcttgagcca
4921 ctgcacctag cctctatttg ttttacaaaa gagaaattga gatcctgaat gttaagtgac
4981 ttgcctgagg ccatcccact aacaggagcc agggttagga ttcaaacccc atccaactgg
5041 tcccagagct ggagcttctt gcactgccct acactaccta ccatctccat cctctgggca
5101 cctttttata agaaccaaaa cattacagag cattgctttg tcaactcagc tgggaacatt
5161 tcccagtgca actcacattt ttcactgctc tgtgcctgtc cgtataagct caatgagtat
5221 tgatttaggg gctttggaga actttgaatg ctacccccca agtaaccatt gttggcaacc
5281 tggtacctct acttttagcc atttctcctt ctctataaat agtgcagaag taacccactt
5341 ggtaacaggc atccttgcca agcctccacc actaggtcag tgtaagaatt aaagaaagag
5401 gaaagaaaca caaaaagtgg cttgatggtt aagacaggtt tattttagag aaaacacacc
5461 tgagaggggc tgctggctga attaggttag agtcttttct acagactaag agtgtttaag
5521 gatttagggt gggagagttt cttagaggct tggactgctt ctgtgttttt tttgttgtgc
5581 ttatatggga gggagagtgg tgtgtttgct tttatacatt tttctgcagc tgtaggcata
5641 ccccccaagt ctgcttttag cttccctatt ttagtgcacc tggagggaaa ggaatgtgct
5701 tattaaggcc cactgtttta ctggggccca ttgtatgagg gtgaagtttg gcagttaccc
5761 aagagacttt tcctccacct tcctctgtgc ccgagctgtt ttatctgcat tttactgtct
5821 gctttttttg gctgcttata gtttttaaaa aagtaatttc cttaaatcca gaaggctaaa
5881 aatgaagctg aaacttaaag tggcggtgtt tgtccaaaat aacggggctc ctgctctgcc
5941 agtcagtacc ctcaagtcac tcctgatcct caacctccat gcctaaggct ggttcaagag
6001 accacataat atctgccttt tattacatac atgatgggtg catgggattc tgcgtgccct
6061 ttgcttgata tagactgcta aggtgagatg gggaatatca gagtcagctg ctgcttgagg
6121 aagcagaaca cacagctgga ggcttggaac atgtgggtcc ctatgagtgt agagcccata
6181 tccccataga gtctacctag agcaggggtc gccaaatgtt ttcttaaaga gcctgatagt
6241 gtatatgtta ggctttgtga gccaggtatt tacagcaact caattctacc actgtggtat
6301 gaaaacagct atagacaatc ataaatgaat gatcatggct atgttttaat aaaactttac
6361 agacactgaa cttgaacttc cattgtgata tgaaaacagc tatagacaat cataaatgaa
6421 tgatcatggc tatgttttaa taaaacttta tggacactga gcttgaaaaa aaaaaaaaaa
6481 aaa
Translation
(SEQ ID NO: 11)
MGGRVFLVFLAFCVWLTLPGAETQDSRGCARWCPQDSSCVNATACRCNPGFSSFSEIITTPMETCDDINE
CATLSKVSCGKFSDCWNTEGSYDCVCSPGYEPVSGAKTFKNESENTCQDVDECQQNPRLCKSYGTCVNTL
GSYTCQCLPGFKLKPEDPKLCTDVNECTSGQNPCHSSTHCLNNVGSYQCRCRPGWQPIPGSPNGPNNTVC
EDVDECSSGQHQCDSSTVCFNTVGSYSCRCRPGWKPRHGIPNNQKDTVCEDMTFSTWTPPPGVHSQTLSR
FFDKVQDLGRDYKPGLANNTIQSILQALDELLEAPGDLETLPRLQQHCVASHLLDGLEDVLRGLSKNLSN
GLLNFSYPAGTELSLEVQKQVDRSVTLRQNQAVMQLDWNQAQKSGDPGPSVVGLVSIPGMGKLLAEAPLV
LEPEKQMLLHETHQGLLQDGSPILLSDVISAFLSNNDTQNLSSPVTFTFSHRSVIPRQKVLCVFWEHGQN
GCGHWATTGCSTIGTRDTSTICRCTHLSSFAVLMAHYDVQEEDPVLTVITYMGLSVSLLCLLLAALTFLL
CKAIQNTSTSLHLQLSLCLFLAHLLFLVAIDQTGHKVLCSIIAGTLHYLYLATLTWMLLEALYLFLTARN
LTVVNYSSINRFMKKLMFPVGYGVPAVTVAISAASRPHLYGTPSRCWLQPEKGFIWGFLGPVCAIFSVNL
VLFLVTLWILKNRLSSLNSEVSTLRNTRMLAFKATAQLFILGCTWCLGILQVGPAARVMAYLFTIINSLQ
GVFIFLVYCLLSQQVREQYGKWSKGIRKLKTESEMHTLSSSAKADTSKPSTVN
```

The regulatory T cells (Tregs), formerly known as suppressor T cells, are a subpopulation of T cells which modulate the immune system, maintain tolerance to self-antigens, and abrogate autoimmune disease. Regulatory T cells come in many forms with the most well-understood being those that express CD4, CD25, and Foxp3 (CD4+CD25+ regulatory T cells, or “Tregs”).

### Compositions

Accordingly, described herein is a composition for treating tumor and/or immune based inflammatory disease comprising an antagonist of a component of EGF-like module-containing mucin-like hormone receptor-like 2 (EMR2) signaling pathway. The antagonist blocks the development of regulatory T cells, tolerogenic macrophages, M2 macrophages, and/or myeloid derived suppressor cells. In addition, the antagonist can block the binding of ERM2 and its ligand.

As described herein, a component of EMR2 signaling pathway can be EMR2 or EMR2 ligand. Exemplary EMR2 ligand includes chondroitin sulfate.

An antagonist of a component of EMR2 signaling pathway can be any composition that blocks either the function (such as binding of EMR2 to its ligand) or the expression of a component of the EMR2 signaling pathway (e.g., a component is EMR2 or EMR2 ligand). For example, an antagonist described herein can be an anti-EMR2 antibody, a soluble EMR2 fragment or a soluble EMR2 ligand fragment, an siRNA against EMR2 or a small molecule. For example, an anti-EMR2 antibody is a monoclonal antibody that binds to the stalk region of EMR2 (e.g., clone 1b5) or an antibody with the same or similar binding specificity of clone 1b5.

An siRNA against EMR2 mRNA (e.g., SEQ ID Nos: 8 or 10) can be designed and synthesized according to any known method in the art. The siRNA sequences of the present invention may have a length of from 5 to 100 nucleotides. The siRNA sequences of the present invention may have a length of at least 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80 or 90 nucleotides. The siRNA sequences of the present invention may be synthesized or expressed in a cell (in vitro or in vivo). Exemplary siRNA sequences against EMR2 mRNA inlcude, but are not limited to, 5′AUGCUGACAUAUUUACUAGAGG (SEQ ID NO: 12); 5′agccgtgtctctcttgatctatgtgt (SEQ ID NO: 13); and 5′ ggcatttgcttggaatatttagg (SEQ ID NO: 14).

For example, an antagonist of EMR2 is an isolated antibody against EMR2 (e.g., SEQ ID NO: 1, 2, 3, 4, 5, 6, 7, 9, or 11) or a fragment thereof. Preferably, the antibody binds to an epitope located in the stalk region of EMR2. The stalk region of EMR2 located within SEQ ID No: 2. The epitope of the antibody can be a fragment or a variant of SEQ ID NO: 2 with a length of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60 or more residues. A “variant polypeptide” means a polypeptide having at least about 60% amino acid sequence identity, more preferably at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% amino acid sequence identity and yet more preferably at least about 99% amino acid sequence identity with an amino acid sequence comprising any one of SEQ ID NOs: 1-7 and 9.

The term “antibody” include polyclonal antibodies, monoclonal antibodies (MAbs), chimeric antibodies, anti-idiotypic (anti-Id) antibodies to antibodies that can be labeled in soluble or bound form, and humanized antibodies as well as fragments thereof provided by any known technique, such as, but not limited to enzymatic cleavage, peptide synthesis or recombinant techniques. The term “antibody” is also meant to include both intact molecules as well as fragments thereof, such as, for example, Fab and F(ab′)₂, which are capable of binding antigen Fab and F(ab′)₂ fragments lack the Fc fragment of intact antibody, clear more rapidly from the circulation, and may have less non-specific tissue binding than an intact antibody (Wahl et al., J. Nucl. Med. 24:316-325 (1983)). An antibody is said to be “capable of binding” a molecule if it is capable of specifically reacting with the molecule. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

Polyclonal antibodies are heterogeneous populations of antibody molecules derived from the sera of animals immunized with an antigen. A monoclonal antibody contains a substantially homogeneous population of antibodies specific to antigens, which population contains substantially similar epitope binding sites. MAbs may be obtained by methods known to those skilled in the art. See, for example Kohler and Milstein, Nature 256:495-497 (1975); U.S. Pat. No. 4,376,110; Ausubel et al, eds., supra, Harlow and Lane, ANTIBODIES: A LABORATORY MANUAL, Cold Spring Harbor Laboratory (1988); and Colligan et al., eds., Current Protocols in Immunology, Greene Publishing Assoc. and Wiley lnterscience, N.Y., (1992, 1993), the contents of which references are incorporated entirely herein by reference. Such antibodies may be of any immunoglobulin class including IgG, IgM, IgE, IgA, GILD and any subclass thereof. A hybridoma producing a MAb of the present invention may be cultivated in vitro, in situ or in vivo. Production of high titers of MAbs in vivo or in situ makes this the presently preferred method of production.

Chimeric antibodies are molecules, different portions of which are derived from different animal species, such as those having the variable region derived from a murine MAb and a human immunoglobulin constant region. Chimeric antibodies are primarily used to reduce immunogenicity in application and to increase yields in production, for example, where murine MAbs have higher yields from hybridomas but higher immunogenicity in humans, such that human/murine chimeric MAbs are used. Chimeric antibodies and methods for their production are known in the art (Cabilly et al, Proc. Natl. Acad. Sci. USA 81:3273-3277 (1984); Morrison et al., Proc. Natl. Acad. Sci. USA 81:6851-6855 (1984); Boulianne et al., Nature 312:643-646 (1984); Cabilly et al., European Patent Application 125023 (published Nov. 14, 1984); Neuberger et al., Nature 314:268-270 (1985); Taniguchi et al., European Patent Application 171496 (published Feb. 19, 1985); Morrison et al., European Patent Application 173494 (published Mar. 5, 1986); Neuberger et al., PCT Application WO 8601533, (published Mar. 13, 1986); Kudo et al., European Patent Application 184187 (published Jun. 11, 1986); Morrison et al., European Patent Application 173494 (published Mar. 5, 1986); Sahagan et al., J. Immunol. 137:1066-1074 (1986); Robinson et al., International Patent Publication, WO 9702671 (published 7 May 1987); Liu et al., Proc. Natl. Acad. Sci. USA 84:3439-3443 (1987); Sun et al., Proc. Natl. Acad. Sci. USA 84:214-218 (1987); Better et al., Science 240:1041-1043 (1988); and Harlow and Lane, ANTIBODIES: A LABORATORY MANUAL, supra. These references are entirely incorporated herein by reference.

An anti-idiotypic (anti-Id) antibody is an antibody, which recognizes unique determinants generally, associated with the antigen-binding site of an antibody. An Id antibody can be prepared by immunizing an animal of the same species and genetic type (e.g., mouse strain) as the source of the MAb with the MAb to which an anti-Id is being prepared. The immunized animal will recognize and respond to the idiotypic determinants of the immunizing antibody by producing an antibody to these idiotypic determinants (the anti-Id antibody). See, for example, U.S. Pat. No. 4,699,880, which is herein entirely incorporated by reference.

The anti-Id antibody may also be used as an “immunogen” to induce an immune response in yet another animal, producing a so-called anti-anti-Id antibody. The anti-anti-Id may be epitopically identical to the original MAb, which induced the anti-Id. Thus, by using antibodies to the idiotypic determinants of a MAb, it is possible to identify other clones expressing antibodies of identical specificity. The term “humanized antibody” is meant to include e.g. antibodies which were obtained by manipulating mouse antibodies through genetic engineering methods so as to be more compatible with the human body. Such humanized antibodies have reduced immunogenicity and improved pharmacokinetics in humans. They may be prepared by techniques known in the art, such as described, e.g. for humanized anti-TNF antibodies in Molecular Immunology, Vol. 30, No. 16, pp. 1443-1453, 1993.

A composition of the invention may include an antagonist that is a soluble fragment of EMR2, which blocks the interaction between EMR2 and its ligand. The fragment may have a length of from 5 to 500, 10 to 100 or 20 to 60 amino acids. The fragment may also have a length of at least 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 260, 280 or 300 amino acids or longer. Amino acid sequences having at least 70% amino acid identity, preferably at least 80% amino acid identity, more preferably at least 90% identity, and most preferably 95% identity to the fragments described herein are also included within the scope of the present invention. Preferably, a soluble fragment or an EMR2-binding fragment is located within SEQ ID NO: 2 with a length of at least 5, 6, 7, 8, 9, 10, 11, 12, 13, 14, 15, 16, 17, 18, 19, 20, 21, 22, 23, 24, 25 or more residues. Preferably, the length of a soluble fragment or an EMR2-binding fragment is 8-10 residues.

The invention further includes a composition for suppressing inflammation in a subject in need thereof, where the composition includes an agonist of EMR2. An agonist is a molecule/compound/peptide that enhances the function (such as binding of EMR2 to its ligand) or expression of EMR2 or other component in the signaling pathway. For example, the agonist is anti-EMR2 monoclonal antibody clone 2A1 (Thermo Scientific, Cat. No. MA5-16474) or an antibody with the binding specificty of clone 2A1.

The present invention also provides pharmaceutical compositions comprising an antagonist of EMR2 or EMR2 ligand or an agonist of EMR2, and optionally in combination with at least one pharmaceutically acceptable excipient or carrier.

A “pharmaceutical composition” is a formulation containing a composition (i.e., an antagonist of EMR2 or EMR2 ligand or an agonist of EMR2) described herein in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., an antagonist of EMR2 or EMR2 ligand or an agonist of EMR2) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a composition of this invention include powders, sprays, ointments, pastes, creams, lotions, gels, solutions, patches and inhalants. In one embodiment, the active composition is mixed under sterile conditions with a pharmaceutically acceptable carrier, and with any preservatives, buffers, or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Preferably, the composition or the pharmaceutical composition described herein is administered locally via injection or via bolus administration, e.g., 10, 50, 100, 250, 500, 1000, 2000 ul of a solution containing an EMR2 modulating composition such as an EMR2 specific antibody (for example, 5 ug/ml or 15 ug/ml or 100 mg/50 mL or 200 mg/100 mL). For example, a composition described herein is formulated in a sustained release formula or delivery vehicle. The vehicle includes dermal patch, an intravenous pump, or another implantable device. The implant is inert, biodegradable, or erodible. For sustained release in an implant, patch, or oral composition, the vehicle contains a semipermeable membrane. The membrane serves the purpose of controlling the rate of delivery of the composition to bodily tissues. In another example of a sustained release formulation, the vehicle contains a plurality particles, each of which are characterized as having a different rate of dissolution. For example, a composition may contain two or more classes of particles: slow, medium, and rapid release particles.

A composition (e.g., an antagonist of EMR2 or EMR2 ligand or an agonist of EMR2) or pharmaceutical composition of the invention can be administered to a subject in many of the well-known methods currently used for treatment of cancer (solid tumors). For example, for treatment of a solid tumor, a composition of the invention may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer) and the health of the patient should preferably be closely monitored.

The pharmaceutical compositions can be included in a container, pack, or dispenser together with instructions for administration.

The dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer or other symptoms of the liver disease that can be treated by the compositions of the instant invention. Dosages can range from about 0.01 ug/kg per day to about 5000 mg/kg per day. For example, dosages can range from about 1 mg/kg per day to about 1000 mg/kg per day. For example, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug interaction(s), reaction sensitivities, and tolerance/response to therapy. Long-acting pharmaceutical compositions may be administered every 3 to 4 days, every week, or once every two weeks depending on half-life and clearance rate of the particular formulation.

### Methods of Treatment

A composition that includes an antagonist, as described herein, can be used for treating or alleviating a symptom of cancer in a subject in need thereof. The method includes administering an effective amount of a composition described herein to the subject, thereby treating or alleviating a symptom of cancer in the subject.

The method may include administering a second agent as part of a specific treatment regimen intended to provide the beneficial effect from the co-action of these therapeutic agents (i.e., combination therapy). For example, the combination therapy includes administration of an effective amount of a composition described herein and an autologous immune enhancement therapy (e.g., administration of an effective amount of T immune cells). Preferably, the autologous immune enhancement therapy is performed 30 min, 1, 2, 3, 4, 5, 6, 12 or 24 hours, 2, 3, 4, 5, 6, 7 or 8 days or longer time after the administration of the composition. A common deficiency of autologous immune enhancement therapy is utilizing excessive amount of cells. A combination therapy can address this problem and reduce the amount of cells used for treatment.

Exemplary cancers that may be treated using a composition described herein include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. Preferably, cancers that can be treated by the composition described herein include solid tumors (e.g., sarcomas, carcinomas or lymphomas), such as bladder carcinoma, breast carcinoma, colorectal carcinoma, gastric and esophageal carcinoma and glioblastoma. For example, the cancer is brain and CNS cancer, kidney cancer, ovarian cancer, pancreatic cancer, lung cancer, breast cancer, colon cancer or prostate cancer.

The compositions including an antagonist described herein can also be used for inhibiting development or for reducing population of myeloid cells that express EMR2. For example, the myeloid cells that express EMR2 are regulatory T cells, suppressor macrophages, tolerogenic macrophages or M2 macrophages. The method includes contacting a cell of interest with a composition described herein. The regulatory T cells express CD4 and CD25. Alternatively, the regulatory T cells express CD8.

A composition including an agonist, as described herein, can be used for a method of suppressing inflammation in a subject in need thereof. The method includes administering the composition to the subject. For example, the subject is suffering from a CD4-mediated inflammatory disease, such as diabetes, Lupus erythematosus, an allergy, rheumatoid arthritis, multiple sclerosis or Crohn's disease. For example, the inflammation is ocular inflammation.

Alternatively, a composition including an agonist, as described herein, can be used for a method of treating or alleviating a symptom of an ocular immune inflammatory disease in a subject in need thereof. The method includes administering to the subject an effective amount of an agonist of EMR2. Exemplary ocular immune inflammatory disease comprises dry eye syndrome, uveitis, corneal ulcer, ocular cicatricial pemphigoid, Mooren's ulcer, scleritis, episcleritis or immunogenic conjunctivitis.

Also provided herein is a method of treating a CD4-mediated inflammatory disease in a subject in need thereof by a) isolating a sample comprising a peripheral blood monocyte cell from the subject; b) incubating the sample with TGF-beta and one of the following: an antigen that is specific for the diseased tissue (target antigen) and an extract of the diseased tissue; and c) administering the incubated sample to the subject, thereby treating the CD4-mediated inflammatory disease. For example, an extract of the diseased tissue is a retinal extract. For example, the CD4-mediated inflammatory disease is diabetes, Lupus erythematosus, an allergy, rheumatoid arthritis, multiple sclerosis or Crohn's disease.

TABLE 1
Exemplary target antigens

| Disease | Target antigen |
|---|---|
| Diabetes mellitus type 1 | Pancreatic beta cell proteins |
| | (possibly insulin, Glutamate |
| | decarboxylase) |
| Multiple sclerosis | Oligodendrocyte proteins |
| | (myelin basic protein, |
| | proteolipid protein) |
| Rheumatoid arthritis | Antigen in synovial membrane |
| | (possibly type II collagen) |
| Some peripheral neuropathies | Schwann cell antigen |
| Hashimoto's Thyroiditis | Thyroglobulin antigen |
| Crohn's disease | Unknown |
| Allergic contact dermatitis | Environmental chemicals, e.g. |
| | poison ivy, nickel |
| Mantoux test* (diagnostic) | Tuberculin |

### Cell Therapy

Further provided herein are vectors, preferably expression vectors, containing nucleic acid sequence encoding EMR2 or fragment thereof.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically.

Another aspect of the invention includes host cells into which a recombinant expression vector of the invention has been introduced.

The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be a premature mammalian cell, i.e., pluripotent stem cell. A host cell can also be derived from other human tissue. For example, a host cell is peripheral blood monocyte. Accordingly, the invention provides an isolated peripheral blood monocyte engineered to express EMR2 or a fragment thereof.

Vector DNA can be introduced into host cells via conventional transformation, transduction, infection or transfection techniques. As used herein, the terms “transformation” “transduction”, “infection” and “transfection” are intended to refer to a variety of art recognized techniques for introducing foreign nucleic acid (e.g., DNA) into a host cell, including calcium phosphate or calcium chloride co precipitation, DEAE dextran mediated transfection, lipofection, or electroporation. In addition transfection can be mediated by a transfection agent. By “transfection agent” is meant to include any compound that mediates incorporation of DNA in the host cell, e.g., liposome. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

Transfection may be “stable” (i.e. integration of the foreign DNA into the host genome) or “transient” (i.e., DNA is episomally expressed in the host cells).

The isolated peripheral blood monocyte engineered to express EMR2 or a fragment thereof described herein can be used for suppressing inflammation in a subject in need thereof. In another example, the method includes administering to the subject an effective amount of patient-drived peripheral blood monocytes that express EMR2 or a fragment thereof. To augument expression of EMR2 in these cells, the patient-drived peripheral blood monocytes are incubated with 1) TGF-beta and a target antigen (e.g., Table 1) or 2) TGF-beta and a diseased tissue extract (for example, a retinal extract) prior to administration to the subject. TGF-beta is human TGF-beta 1 or TGF-beta 2.

Inflammation that can be treated by the engineered peripheral blood monocytes can be ocular inflammation. In addition, the subject may be suffering from a CD4-mediated inflammatory disease, such as diabetes, Lupus erythematosus, an allergy, rheumatoid arthritis, multiple sclerosis or Crohn's disease.

As the essential organ providing vision to living animals, eyes displays an important trait called ocular immune privilege, which allows it to coexist with the external environment without immune collateral damage (Abbas & Lichtman, n.d.). Many of the eye's cells are permanent resident cells that rarely undergo regeneration. Thus, inflammation and its products could be irreversibly damaging and counteractive for the survival of the eye (Stein-Streilein & Lucas, 2011).

### Kits

A composition of the present invention may, if desired, be presented in a kit (e.g., a pack or dispenser device) which may contain one or more unit dosage forms containing the composition.

The pack may for example comprise metal or plastic foil, such as a blister pack. The pack or dispenser device may be accompanied by instructions for administration. Compositions comprising a composition of the invention formulated in a compatible pharmaceutical carrier may also be prepared, placed in an appropriate container, and labeled for treatment of an indicated condition. Instructions for use may also be provided.

### REFERENCES

Abbas, Abul K.; Lichtman, Andrew H. (n.d.). Basic Immunology: Functions and Disorders of the Immune System (4th ed.). Philadelphia, Pa., London: Saunders/Elsevier.
Abbott, R. J. M., Spendlove, I., Roversi, P., Fitzgibbon, H., Knott, V., Teriete, P., . . . Lea, S. M. (2007). Structural and Functional Characterization of a Novel T Cell Receptor Co-regulatory Protein Complex, CD97-CD55. Journal of Biological Chemistry, 282(30), 22023-22032. doi:10.1074/jbc.M702588200
Caminschi, I., Vandenabeele, S., Sofi, M., McKnight, A. J., Ward, N., Brodnicki, T. C., . . . Wright, M. D. (2006). Gene structure and transcript analysis of the human and mouse EGF-TM7 molecule, FIRE. DNA Sequence: The Journal of DNA Sequencing and Mapping, 17(1), 8-14. doi:10.1080/10425170500355737
Cella, M., Dohring, C., Samaridis, J., Dessing, M., Brockhaus, M., Lanzavecchia, A., & Colonna, M. (1997). A Novel Inhibitory Receptor (ILT3) Expressed on Monocytes, Macrophages, and Dendritic Cells Involved in Antigen Processing. The Journal of Experimental Medicine, 185(10), 1743-1751. doi:10.1084/jem.185.10.1743
D'Orazio, T. J., & Niederkorn, J. Y. (1998). A Novel Role for TGF-β and IL-10 in the Induction of Immune Privilege. The Journal of Immunology, 160(5), 2089-2098.
Davies, Lin, H.-H., Stacey, M., Yona, S., Chang, G.-W., Gordon, S., . . . Fox, S. B. (2011). Leukocyte adhesion-GPCR EMR2 is aberrantly expressed in human breast carcinomas and is associated with patient survival. Oncology Reports, 25(3). doi:10.3892/or.2010.1117
EMR4P egf-like module containing, mucin-like, hormone receptor-like 4 pseudogene [Homo sapiens (human)]—Gene—NCBI. (n.d.). Retrieved Mar. 4, 2014, from http://www.ncbi.nlm.nih.gov/gene/326342
Faunce, D. E., Sonoda, K.-H., & Stein-Streilein, J. (2001). MIP-2 Recruits NKT Cells to the Spleen During Tolerance Induction. The Journal of Immunology, 166(1), 313-321.
Forrester, J. V., & Xu, H. (2012). Good news-bad news: the Yin and Yang of immune privilege in the eye. Frontiers in Immunological Tolerance, 3, 338. doi:10.3389/fimmu.2012.00338
Gregory, M. S., Hackett, C. G., Abernathy, E. F., Lee, K. S., Saff, R. R., Hohlbaum, A. M., . . . Ksander, B. R. (2011). Opposing Roles for Membrane Bound and Soluble Fas Ligand in Glaucoma-Associated Retinal Ganglion Cell Death. PLoS ONE, 6(3), e17659. doi:10.1371/journal.pone.0017659
Hamann, J., Koning, N., Pouwels, W., Ulfman, L. H., van Eijk, M., Stacey, M., . . . Kwakkenbos, M. J. (2007). EMR1, the human homolog of F4/80, is an eosinophil-specific receptor. European Journal of Immunology, 37(10), 2797-2802. doi:10.1002/eji.200737553 Ji, S.-X., Yin, X.-L., & Yang, P.-Z. (2011). Effect of CD4+CD25+ regulatory T cells in the development of anterior chamber-associated immune deviation. International Journal of Ophthalmology, 4(1), 19-25. doi:10.3980/j.issn.2222-3959.2011.01.04
Kaplan, H. J., & Streilein, J. W. (1977). Immune Response to Immunization Via the Anterior Chamber of the Eye I. F1 Lymphocyte-Induced Immune Deviation. The Journal of Immunology, 118(3), 809-814.
Kaplan, H. J., & Streilein, J. W. (1978). Immune Response to Immunization via the Anterior Chamber of the Eye II. An Analysis of F1 Lymphocyte-Induced Immune Deviation. The Journal of Immunology, 120(3), 689-693.
Lin, H.-H., Faunce, D. E., Stacey, M., Terajewicz, A., Nakamura, T., Zhang-Hoover, J., . . . Stein-Streilein, J. (2005). The macrophage F4/80 receptor is required for the induction of antigen-specific efferent regulatory T cells in peripheral tolerance. The Journal of Experimental Medicine, 201(10), 1615-1625. doi:10.1084/jem.20042307
Lin, H.-H., Stacey, M., Hamann, J., Gordon, S., & McKnight, A. J. (2000). Human EMR2, a Novel EGF-TM7 Molecule on Chromosome 19p13.1, Is Closely Related to CD97. Genomics, 67(2), 188-200. doi:10.1006/geno.2000.6238
Lucas, K., Karamichos, D., Mathew, R., Zieske, J. D., & Stein-Streilein, J. (2012). Retinal Laser Burn-Induced Neuropathy Leads to Substance P-Dependent Loss of Ocular Immune Privilege. The Journal of Immunology, 189(3), 1237-1242. doi:10.4049/jimmunol.1103264 McKnight, A. J., & Gordon, S. (1998). The EGF-TM7 family: unusual structures at the leukocyte surface. Journal of Leukocyte Biology, 63(3), 271-280.
Niederkorn, J. Y., & Streilein, J. W. (1983). Alloantigens placed into the anterior chamber of the eye induce specific suppression of delayed-type hypersensitivity but normal cytotoxic T lymphocyte and helper T lymphocyte responses. The Journal of Immunology, 131(6), 2670-2674.
Schmitt, E. G., & Williams, C. B. (2013). Generation and function of induced regulatory T cells. Immunological Tolerance, 4, 152. doi:10.3389/fimmu.2013.00152
Shao, H., Kaplan, H. J., & Sun, D. (2007). Expression of MHC molecules by parenchymal cells of the target organ in autoimmune disease decreases the detrimental effect of invading autoreactive T cells. Chemical Immunology and Allergy, 92, 94-104. doi: 10.1159/000099260
Sonoda, K.-H., Faunce, D. E., Taniguchi, M., Exley, M., Balk, S., & Stein-Streilein, J. (2001). NK T Cell-Derived IL-10 Is Essential for the Differentiation of Antigen-Specific T Regulatory Cells in Systemic Tolerance. The Journal of Immunology, 166(1), 42-50.
Stacey, M., Chang, G.-W., Davies, J. Q., Kwakkenbos, M. J., Sanderson, R. D., Hamann, J., . . . Lin, H.-H. (2003). The epidermal growth factor-like domains of the human EMR2 receptor mediate cell attachment through chondroitin sulfate glycosaminoglycans. Blood, 102(8), 2916-2924. doi:10.1182/blood-2002-11-3540
Stacey, M., Lin, H.-H., Hilyard, K. L., Gordon, S., & McKnight, A. J. (2001). Human Epidermal Growth Factor (EGF) Module-containing Mucin-like Hormone Receptor 3 Is a New Member of the EGF-TM7 Family That Recognizes a Ligand on Human Macrophages and Activated Neutrophils. Journal of Biological Chemistry, 276(22), 18863-18870. doi:10.1074/jbc.M101147200
Stein-Streilein, J. (2006). Characterization of Dendritic Cells and other Antigen-presenting Cells in the Eye. In Handbook of Dendritic Cells (Vol. 1, pp. 101-118). Wiley-VCH.
Stein-Streilein, J., & Lucas, K. (2011). A Current Understanding of Ocular Immune Privilege. Current Immunology Reviews, 7(3), 336-343. doi:10.2174/157339511796196683
Streilein, J. W., Masli, S., Takeuchi, M., & Kezuka, T. (2002). The eye's view of antigen presentation. Human Immunology, 63(6), 435-443. doi:10.1016/S0198-8859(02)00393-2
Streilein, J. W., & Niederkorn, J. Y. (2007). Induction of Anterior Chamber-Associated Immune Deviation Requires an Intact, Functional Spleen*. Ocular Immunology and Inflammation, 15(3), 187-194. doi:10.1080/09273940701382473
Takeuchi, M., Keino, H., Suzuki, J., Usui, Y., Hattori, T., Takeuchi, A., . . . Usui, M. (2006). Possibility of inducing anterior chamber-associated immune deviation by TGF-β2 treatment of monocytes isolated from Behcet's patients. Experimental Eye Research, 83(4), 981-988. doi:10.1016/j.exer.2006.05.005
Taylor, A. W. (2007). Ocular Immunosuppressive Microenvironment. In J. Y. Niederkorn & H. J. Kaplan (Eds.), Chemical Immunology and Allergy (pp. 71-85). Basel: KARGER. Retrieved from http://www.karger.com.ezproxy.bu.edu/Article/Abstract/99255
Willbanks, G. A., Rootman, D. S., Jay, V., Wiggert, B., Chamberlain, J., & Inman, R. D. (1997). Experimental autoimmune uveitis in HLA-B27 transgenic mice. Human Immunology, 53(2), 188-194. doi:10.1016/S0198-8859(97)00033-5

What is claimed:

1. A composition for treating a tumor or an immune based inflammatory disease comprising an antagonist of a component of EGF-like module-containing mucin-like hormone receptor-like 2 (EMR2) signaling pathway, wherein the antagonist inhibits EMR2-expressing myeloid cells.

2. The composition of claim 1, wherein the myeloid cells expressing EMR2 comprise regulatory T cells.

3. The composition of claim 1, wherein the component comprises EMR2.

4. The composition of claim 1, wherein the component comprises an EMR2 ligand.

5. The composition of claim 4, wherein the EMR2 ligand comprises chondroitin sulfate.

6. The composition of claim 1, wherein the antagonist comprises an anti-EMR2 antibody or EMR2-binding fragment thereof.

7. The composition of claim 6, wherein the anti-EMR2 antibody comprises a monoclonal antibody.

8. The composition of claim 7, wherein the anti-EMR2 antibody comprises a humanized monoclonal antibody.

9. The composition of claim 1, wherein the antagonist comprises an siRNA against EMR2.

10. The composition of claim 1, wherein the antagonist comprises a small peptide molecule.

11. The composition of claim 1, wherein the antagonist comprises a small molecule.

12. A method of treating or alleviating a symptom of cancer in a subject in need thereof, comprising administering to the subject an effective amount of the composition of claim 1.

13. The method of claim 12, wherein the cancer comprises a solid tumor.

14. The method of claim 13, wherein the solid tumor comprises sarcoma, carcinoma or lymphoma.

15. The method of claim 12, where in the cancer comprises brain and CNS cancer, kidney cancer, ovarian cancer, pancreatic cancer, lung cancer, breast cancer, colon cancer or prostate cancer.

16. The method of claim 12, further comprising performing an autologous immune enhancement therapy.

17. The method of claim 16, wherein the autologous immune enhancement therapy comprises administering to the subject an effective amount of autologous T immune cells.

18. A method of inhibiting development or reducing a population of myeloid cells expressing EMR2, the method comprising contacting a myeloid cell expressing EMR2 with the composition of claim 1.

19. The method of claim 18, wherein the myeloid cells expressing EMR2 comprise regulatory T cells.

20. The method of claim 19, wherein the regulatory T cells express CD4 and CD25.

21. An isolated antibody against EMR2 or a F4/80 or fragments thereof.

22. The antibody of claim 21, wherein the antibody comprises a monoclonal antibody, a polyclonal antibody, a chimeric antibody or a single-chain antibody.

23. The antibody of claim 22, wherein the antibody comprises a humanized monoclonal antibody.

24. The antibody of claim 22, wherein the antibody binds to an epitope located in the stalk region of EMR2.

25. An isolated peripheral blood monocyte engineered to express EMR2.

26. A method of suppressing inflammation in a subject in need thereof, the method comprising administering to the subject an effective amount of the isolated peripheral blood monocyte of claim 25.

27. The method of claim 26, further comprising incubating the peripheral blood monocyte with TGF-beta and a tissue extraction prior to administration.

28. The method of claim 27, wherein the subject suffers from a CD4-mediated inflammatory disease.

29. The method of claim 28, wherein the CD4-mediated inflammatory disease comprises diabetes, Lupus erythematosus, an allergy, rheumatoid arthritis, multiple sclerosis or Crohn's disease.

30. The method of claim 27, wherein the TGF-beta comprises TGF-beta 1 or TGF-beta 2.

31. The method of claim 26, wherein the inflammation comprises ocular inflammation.

32. A method of treating or alleviating a symptom of an ocular immune inflammatory disease in a subject in need thereof, the method comprising administering to the subject a therapeutically effective amount of an agonist of EMR.

33. The method of claim 32, wherein the ocular immune inflammatory disease comprises dry eye syndrome, uveitis, corneal ulcer, ocular cicatricial pemphigoid, Mooren's ulcer, scleritis, episcleritis or immunogenic conjunctivitis.

34. A method of treating a CD4-mediated inflammatory disease in a subject in need thereof, comprising
a) isolating a sample comprising a peripheral blood monocyte cell from the subject;
b) incubating the sample with TGF-beta and
i) an antigen that is specific for the diseased tissue or
ii) an extract of the diseased tissue; and
c) administering the incubated sample to the subject, thereby treating the CD4-mediated inflammatory disease.

35. The method of claim 34, wherein the CD4-mediated inflammatory disease comprises diabetes, Lupus erythematosus, an allergy, rheumatoid arthritis, multiple sclerosis or Crohn's disease.


SEQUENCE LISTING (14 sequences)

SEQ ID NO: 1 - protein, 823 aa, Homo sapiens
MGGRVFLVFLAFCVWLTLPGAETQDSRGCARWCPQDSSCVNATACRCNPGFSSFSEIITTPMETCDDINECATLSKVSCGKFSDCWNTEGSYDCVCSPGYEPVSGAKTFKNESENTCQDVDECQQNPRLCKSYGTCVNTLGSYTCQCLPGFKLKPEDPKLCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 2 - protein, 267 aa, Homo sapiens
DECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKV

SEQ ID NO: 3 - protein, 812 aa, Homo sapiens
MGGRVFLVFLAFCVWLTLPGAETQDSRGCARWCPQDSSCVNATACRCNPGFSSFSEIITTPMETCDDINECATLSKVSCGKFSDCWNTEGSYDCVCSPGYEPVSGAKTFKNESENTCQDVDECQQNPRLCKSYGTCVNTLGSYTCQCLPGFKLKPEDPKLCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 4 - protein, 774 aa, Homo sapiens
MGGRVFLVFLAFCVWLTLPGAETQDSRGCARWCPQDSSCVNATACRCNPGFSSFSEIITTPMETCDDINECATLSKVSCGKFSDCWNTEGSYDCVCSPGYEPVSGAKTFKNESENTCQDVDECQQNPRLCKSYGTCVNTLGSYTCQCLPGFKLKPEDPKLCTDVDECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 5 - protein, 730 aa, Homo sapiens
MGGRVFLVFLAFCVWLTLPGAETQDSRGCARWCPQDSSCVNATACRCNPGFSSFSEIITTPMETCDDINECATLSKVSCGKFSDCWNTEGSYDCVCSPGYEPVSGAKTFKNESENTCQDVDECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 6 - protein, 681 aa, Homo sapiens
MGGRVFLVFLAFCVWLTLPGAETQDSRGCARWCPQDSSCVNATACRCNPGFSSFSEIITTPMETCDDINECATLSKVSCGKFSDCWNTEGSYDCVCSPGYEPVSGAKTFKNESENTCQDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 7 - protein, 765 aa, Homo sapiens
MGGRVFLVFLAFCVWLTLPGAETQDSRGCARWCPQDSSCVNATACRCNPGFSSFSEIITTPMETCDDINECATLSKVSCGKFSDCWNTEGSYDCVCSPGYEPVSGAKTFKNESENTCQDVDECQQNPRLCKSYGTCVNTLGSYTCQCLPGFKLKPEDPKLCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHREEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 8 - DNA, 6024 bp, Homo sapiens
ctgcaccgccagttccggggagggccctgggccagcggctgtccgccccccctcctttataaagtcctggcctcgggacagcccgcacagctgcccagcctgcggagacgggacagccctgtcccactcactctttcccctgctgctcctgccggcagctcagctggaaccatgggaggccgcgtctttctcgtctttctcgcattctgtgtctggctgactctgccgggagctgaaacccaggactccaggggctgtgcccggtggtgccctcaggactcctcgtgtgtcaatgccaccgcctgtcgctgcaatccagggttcagctctttttctgagatcatcaccacccccatggagacttgtgacgacatcaacgagtgtgcaacactgtcgaaagtgtcatgcggaaaattctcggactgctggaacacagaggggagctacgactgcgtgtgcagcccaggatatgagcctgtttctggggcaaaaacattcaagaatgagagcgagaacacgtgtcaagatgtggacgaatgtcagcagaacccaaggctctgtaaaagctacggcacctgcgtcaacaccctcggcagctacacgtgccagtgcctgcctggcttcaagctcaaacctgaggacccgaagctctgcacagatgtgaatgaatgcacctccggacaaaacccatgccacagctccacccactgcctcaacaacgtgggcagctatcagtgccgctgccgcccgggctggcaaccgattccggggtcccccaatggcccaaacaataccgtctgtgaagatgtggacgagtgcagctccgggcagcatcagtgtgacagctccaccgtctgcttcaacaccgtgggttcatacagctgccgctgccgcccaggctggaagcccagacacggaatcccgaataaccaaaaggacactgtctgtgaagatatgactttctccacctggaccccgccccctggagtccacagccagacgctttcccgattcttcgacaaagtccaggacctgggcagagactacaagccaggcttggccaataacaccatccagagcatcttacaggcgctggatgagctgctggaggcccctggggacctggagaccctgccccgcttacagcagcactgtgtggccagtcacctgctggatggcctagaggatgtcctcagaggcctgagcaagaacctttccaatgggctgttgaacttcagttatcctgcaggcacagaattgtccctggaggtgcagaagcaagtagacaggagtgtcaccttgagacagaatcaggcagtgatgcagctcgactggaatcaggcacagaaatctggtgacccaggcccttctgtggtgggccttgtctccattccagggatgggcaagttgctggctgaggcccctctggtcctggaacctgagaagcagatgcttctgcatgagacacaccagggcttgctgcaggacggctcccccatcctgctctcagatgtgatctctgcctttctgagcaacaacgacacccaaaacctcagctccccagttaccttcaccttctcccaccgtgaggaggatcccgtgctgactgtcatcacctacatggggctgagcgtctctctgctgtgcctcctcctggcggccctcacttttctcctgtgtaaagccatccagaacaccagcacctcactgcatctgcagctctcgctctgcctcttcctggcccacctcctcttcctcgtggcaattgatcaaaccggacacaaggtgctgtgctccatcatcgccggtaccttgcactatctctacctggccaccttgacctggatgctgctggaggccctgtacctcttcctcactgcacggaacctgacggtggtcaactactcaagcatcaacagattcatgaagaagctcatgttccctgtgggctacggagtcccagctgtgacagtggccatttctgcagcctccaggcctcacctttatggaacaccttcccgctgctggctccaaccagaaaagggatttatatggggcttccttggacctgtctgcgccatcttctctgtgaatttagttctctttctggtgactctctggattttgaaaaacagactctcctccctcaatagtgaagtgtccaccctccggaacacaaggatgctggcatttaaagcgacagctcagctgttcatcctgggctgcacgtggtgtctgggcatcttgcaggtgggtccggctgcccgggtcatggcctacctcttcaccatcatcaacagcctgcagggtgtcttcatcttcctggtgtactgcctcctcagccagcaggtccgggagcaatatgggaaatggtccaaagggatcaggaaattgaaaactgagtctgagatgcacacactctccagcagtgctaaggctgacacctccaaacccagcacggttaactagaaaaatcttctgaataagatcttccctctttgcccgtggaaaatctgaacaatctttgagccatctagaggggaaagaaaagactttgttctgtgtgtttcaagaaattcaccatgtcagcaatatgaaggatgttatggaaggcgtgctaggcattcaattcctgcagaaaccggaaatcttccatgccctgcaatgtgctcatcaaactctcagcatatggacggccagctgtggcccatatcttggtcactctgaagcacaatatttatgaagctatagaacgttaagacctctttcacagcctctccttcctacaaagactcctccaaatcttaaaatgaagcaggaaaacgagcctaagaggactttcataccgacaacatctgaaaggactagaatgttcacaccacgatctggatttcttaattttttgtttttgtttttgttgttctctagttctacgggtttgattatttagtcatgtgaaaaatattgattactcacacatagatcaagagagacacggctcctgccttcatggagcttttaggggaaaatgaagtggctcttgcagctagagttgactcagaagccgaaattcctagaaatcaggtttctactgctaggcaattgaagtataaactattttataaacactgtcttctttcgtcttcacaccaacatgcagaaaagtttctaatctcagatcggggatgtgcaacaaattccatttcaaaggaatgacctgcaaaactcctaaatattccaagcaaatgcccttaaccctgtctgttatctgctttccttgaacagaaattctacatgaccataaaacctcgaagatgggtatggcacagttcatgccctgtaatcctagcactttgggagggtgaggcaggaggatggctcaagcccaggagtttgagaccagtgtgggcaacagagtgagaaccatctctacccaaaaaaaaaattaaaaattagccaagcatggtgatgatataggagttaaggagaaatcatttaggcaaatagcaagggtaggaagtcctcagtaaggttttccatttaatgaaaagcagcccccaaaatcattttcttttctaacaaagaacagcctgtaaaatcgagctgcagacatagacaagcaagctggaagcttccacgggtgaatgccggcagctgtgccaataggaaaaagctacctagactaggcatgtccaaaatggcggctccaagttcccttctctttgccagccatgtgtacagtaaaaagcaggcaacatagtgtcagccaaagctcatttgcataataagattagggtggggtggccagctcacataggggtaggccctaggtaaatcagacaccgccttctcaagcctgtctataaaatctggtacactatgacgagggtcagatttcccattcagacgcccctctcccatgcaagagaaagagctgttctcctttctctttcttttgcctattaaacctctgctcctggccaggcacagtggctcacgcctataatcccagcactttgggaggctgaggtggtcagatcacctaaggtcaggagttcaagaccagcctggtcaacatggtgaaatcttgtctctagtaaaaatacaaaaatatatgaaatctcacatagatgataatattaagttccaaaagcaactcaacctggtagattctaattttttttgaggcagggtcttgctttgtcacccatgctggagtacaatggcacaaacactgctcactgcagcctcgacctcccaaggcctaagcaatcctcctgcctcagtccccctccaggtatttgaaactacaggtgtgtaccaccacaccgggctaatttttgtattttttgtagagacgtgggtctcactatgctgcccaggctcaggtcttaatctcctgagctcaggcaatccgcaggcctcagcctccctaagtgcggggattacaggcttgagccactgcacctagcctctatttgttttacaaaagagaaattgagatcctgaatgttaagtgacttgcctgaggccatcccactaacaggagccagggttaggattcaaaccccatccaactggtcccagagctggagcttcttgcactgccctacactacctaccatctccatcctctgggcacctttttataagaaccaaaacattacagagcattgctttgtcaactcagctgggaacatttcccagtgcaactcacatttttcactgctctgtgcctgtccgtataagctcaatgagtattgatttaggggctttggagaactttgaatgctaccccccaagtaaccattgttggcaacctggtacctctacttttagccatttctccttctctataaatagtgcagaagtaacccacttggtaacaggcatccttgccaagcctccaccactaggtcagtgtaagaattaaagaaagaggaaagaaacacaaaaagtggcttgatggttaagacaggtttattttagagaaaacacacctgagaggggctgctggctgaattaggttagagtcttttctacagactaagagtgtttaaggatttagggtgggagagtttcttagaggcttggactgcttctgtgttttttttgttgtgcttatatgggagggagagtggtgtgtttgcttttatacatttttctgcagctgtaggcataccccccaagtctgcttttagcttccctattttagtgcacctggagggaaaggaatgtgcttattaaggcccactgttttactggggcccattgtatgagggtgaagtttggcagttacccaagagacttttcctccaccttcctctgtgcccgagctgttttatctgcattttactgtctgctttttttggctgcttatagtttttaaaaaagtaatttccttaaatccagaaggctaaaaatgaagctgaaacttaaagtggcggtgtttgtccaaaataacggggctcctgctctgccagtcagtaccctcaagtcactcctgatcctcaacctccatgcctaaggctggttcaagagaccacataatatctgccttttattacatacatgatgggtgcatgggattctgcgtgccctttgcttgatatagactgctaaggtgagatggggaatatcagagtcagctgctgcttgaggaagcagaacacacagctggaggcttggaacatgtgggtccctatgagtgtagagcccatatccccatagagtctacctagagcaggggtcgccaaatgttttcttaaagagcctgatagtgtatatgttaggctttgtgagccaggtatttacagcaactcaattctaccactgtggtatgaaaacagctatagacaatcataaatgaatgatcatggctatgttttaataaaactttacagacactgaacttgaacttccattgtgatatgaaaacagctatagacaatcataaatgaatgatcatggctatgttttaataaaactttatggacactgagcttgaaaaaaaaaaaaaaaaaa

SEQ ID NO: 9 - protein, 765 aa, Homo sapiens; identical to SEQ ID NO: 7

SEQ ID NO: 10 - DNA, 6483 bp, Homo sapiens
ggggggtctgctctgtgcggtgaagcttctcttcttggcacctgcctggcatcggaagagggccccttctccctccctgggcttttatgtggacactgtaatgcctcagttttctttctttctttttgtttttgacacagggtctcactctatcacccaagctggagtgcagtggcaccatcttagctcaccgcagcctcaaactccccagctcgggtgattcttcctgcctcagcctcctgagtagtagcagctgggaccacagacgtctgccaccaagcccagctgcaccgccagttccggggagggccctgggccagcggctgtccgccccccctcctttataaagtcctggcctcgggacagcccgcacagctgcccagcctgcggagacgggacagccctgtcccactcactctttcccctgctgctcctgccggcagctcagctggaaccatgggaggccgcgtctttctcgtctttctcgcattctgtgtctggctgactctgccgggagctgaaacccaggactccaggggctgtgcccggtggtgccctcaggactcctcgtgtgtcaatgccaccgcctgtcgctgcaatccagggttcagctctttttctgagatcatcaccacccccatggagacttgtgacgacatcaacgagtgtgcaacactgtcgaaagtgtcatgcggaaaattctcggactgctggaacacagaggggagctacgactgcgtgtgcagcccaggatatgagcctgtttctggggcaaaaacattcaagaatgagagcgagaacacgtgtcaagatgtggacgaatgtcagcagaacccaaggctctgtaaaagctacggcacctgcgtcaacaccctcggcagctacacgtgccagtgcctgcctggcttcaagctcaaacctgaggacccgaagctctgcacagatgtgaatgaatgcacctccggacaaaacccatgccacagctccacccactgcctcaacaacgtgggcagctatcagtgccgctgccgcccgggctggcaaccgattccggggtcccccaatggcccaaacaataccgtctgtgaagatgtggacgagtgcagctccgggcagcatcagtgtgacagctccaccgtctgcttcaacaccgtgggttcatacagctgccgctgccgcccaggctggaagcccagacacggaatcccgaataaccaaaaggacactgtctgtgaagatatgactttctccacctggaccccgccccctggagtccacagccagacgctttcccgattcttcgacaaagtccaggacctgggcagagactacaagccaggcttggccaataacaccatccagagcatcttacaggcgctggatgagctgctggaggcccctggggacctggagaccctgccccgcttacagcagcactgtgtggccagtcacctgctggatggcctagaggatgtcctcagaggcctgagcaagaacctttccaatgggctgttgaacttcagttatcctgcaggcacagaattgtccctggaggtgcagaagcaagtagacaggagtgtcaccttgagacagaatcaggcagtgatgcagctcgactggaatcaggcacagaaatctggtgacccaggcccttctgtggtgggccttgtctccattccagggatgggcaagttgctggctgaggcccctctggtcctggaacctgagaagcagatgcttctgcatgagacacaccagggcttgctgcaggacggctcccccatcctgctctcagatgtgatctctgcctttctgagcaacaacgacacccaaaacctcagctccccagttaccttcaccttctcccaccgttcagtgatcccgagacagaaggtgctctgtgtcttctgggagcatggccagaatggatgtggtcactgggccaccacaggctgcagcacaataggcaccagagacaccagcaccatctgccgttgcacccacctgagcagctttgccgtcctcatggcccactacgatgtgcaggaggaggatcccgtgctgactgtcatcacctacatggggctgagcgtctctctgctgtgcctcctcctggcggccctcacttttctcctgtgtaaagccatccagaacaccagcacctcactgcatctgcagctctcgctctgcctcttcctggcccacctcctcttcctcgtggcaattgatcaaaccggacacaaggtgctgtgctccatcatcgccggtaccttgcactatctctacctggccaccttgacctggatgctgctggaggccctgtacctcttcctcactgcacggaacctgacggtggtcaactactcaagcatcaacagattcatgaagaagctcatgttccctgtgggctacggagtcccagctgtgacagtggccatttctgcagcctccaggcctcacctttatggaacaccttcccgctgctggctccaaccagaaaagggatttatatggggcttccttggacctgtctgcgccatcttctctgtgaatttagttctctttctggtgactctctggattttgaaaaacagactctcctccctcaatagtgaagtgtccaccctccggaacacaaggatgctggcatttaaagcgacagctcagctgttcatcctgggctgcacgtggtgtctgggcatcttgcaggtgggtccggctgcccgggtcatggcctacctcttcaccatcatcaacagcctgcagggtgtcttcatcttcctggtgtactgcctcctcagccagcaggtccgggagcaatatgggaaatggtccaaagggatcaggaaattgaaaactgagtctgagatgcacacactctccagcagtgctaaggctgacacctccaaacccagcacggttaactagaaaaatcttctgaataagatcttccctctttgcccgtggaaaatctgaacaatctttgagccatctagaggggaaagaaaagactttgttctgtgtgtttcaagaaattcaccatgtcagcaatatgaaggatgttatggaaggcgtgctaggcattcaattcctgcagaaaccggaaatcttccatgccctgcaatgtgctcatcaaactctcagcatatggacggccagctgtggcccatatcttggtcactctgaagcacaatatttatgaagctatagaacgttaagacctctttcacagcctctccttcctacaaagactcctccaaatcttaaaatgaagcaggaaaacgagcctaagaggactttcataccgacaacatctgaaaggactagaatgttcacaccacgatctggatttcttaattttttgtttttgtttttgttgttctctagttctacgggtttgattatttagtcatgtgaaaaatattgattactcacacatagatcaagagagacacggctcctgccttcatggagcttttaggggaaaatgaagtggctcttgcagctagagttgactcagaagccgaaattcctagaaatcaggtttctactgctaggcaattgaagtataaactattttataaacactgtcttctttcgtcttcacaccaacatgcagaaaagtttctaatctcagatcggggatgtgcaacaaattccatttcaaaggaatgacctgcaaaactcctaaatattccaagcaaatgcccttaaccctgtctgttatctgctttccttgaacagaaattctacatgaccataaaacctcgaagatgggtatggcacagttcatgccctgtaatcctagcactttgggagggtgaggcaggaggatggctcaagcccaggagtttgagaccagtgtgggcaacagagtgagaaccatctctacccaaaaaaaaaattaaaaattagccaagcatggtgatgatataggagttaaggagaaatcatttaggcaaatagcaagggtaggaagtcctcagtaaggttttccatttaatgaaaagcagcccccaaaatcattttcttttctaacaaagaacagcctgtaaaatcgagctgcagacatagacaagcaagctggaagcttccacgggtgaatgccggcagctgtgccaataggaaaaagctacctagactaggcatgtccaaaatggcggctccaagttcccttctctttgccagccatgtgtacagtaaaaagcaggcaacatagtgtcagccaaagctcatttgcataataagattagggtggggtggccagctcacataggggtaggccctaggtaaatcagacaccgccttctcaagcctgtctataaaatctggtacactatgacgagggtcagatttcccattcagacgcccctctcccatgcaagagaaagagctgttctcctttctctttcttttgcctattaaacctctgctcctggccaggcacagtggctcacgcctataatcccagcactttgggaggctgaggtggtcagatcacctaaggtcaggagttcaagaccagcctggtcaacatggtgaaatcttgtctctagtaaaaatacaaaaatatatgaaatctcacatagatgataatattaagttccaaaagcaactcaacctggtagattctaattttttttgaggcagggtcttgctttgtcacccatgctggagtacaatggcacaaacactgctcactgcagcctcgacctcccaaggcctaagcaatcctcctgcctcagtccccctccaggtatttgaaactacaggtgtgtaccaccacaccgggctaatttttgtattttttgtagagacgtgggtctcactatgctgcccaggctcaggtcttaatctcctgagctcaggcaatccgcaggcctcagcctccctaagtgcggggattacaggcttgagccactgcacctagcctctatttgttttacaaaagagaaattgagatcctgaatgttaagtgacttgcctgaggccatcccactaacaggagccagggttaggattcaaaccccatccaactggtcccagagctggagcttcttgcactgccctacactacctaccatctccatcctctgggcacctttttataagaaccaaaacattacagagcattgctttgtcaactcagctgggaacatttcccagtgcaactcacatttttcactgctctgtgcctgtccgtataagctcaatgagtattgatttaggggctttggagaactttgaatgctaccccccaagtaaccattgttggcaacctggtacctctacttttagccatttctccttctctataaatagtgcagaagtaacccacttggtaacaggcatccttgccaagcctccaccactaggtcagtgtaagaattaaagaaagaggaaagaaacacaaaaagtggcttgatggttaagacaggtttattttagagaaaacacacctgagaggggctgctggctgaattaggttagagtcttttctacagactaagagtgtttaaggatttagggtgggagagtttcttagaggcttggactgcttctgtgttttttttgttgtgcttatatgggagggagagtggtgtgtttgcttttatacatttttctgcagctgtaggcataccccccaagtctgcttttagcttccctattttagtgcacctggagggaaaggaatgtgcttattaaggcccactgttttactggggcccattgtatgagggtgaagtttggcagttacccaagagacttttcctccaccttcctctgtgcccgagctgttttatctgcattttactgtctgctttttttggctgcttatagtttttaaaaaagtaatttccttaaatccagaaggctaaaaatgaagctgaaacttaaagtggcggtgtttgtccaaaataacggggctcctgctctgccagtcagtaccctcaagtcactcctgatcctcaacctccatgcctaaggctggttcaagagaccacataatatctgccttttattacatacatgatgggtgcatgggattctgcgtgccctttgcttgatatagactgctaaggtgagatggggaatatcagagtcagctgctgcttgaggaagcagaacacacagctggaggcttggaacatgtgggtccctatgagtgtagagcccatatccccatagagtctacctagagcaggggtcgccaaatgttttcttaaagagcctgatagtgtatatgttaggctttgtgagccaggtatttacagcaactcaattctaccactgtggtatgaaaacagctatagacaatcataaatgaatgatcatggctatgttttaataaaactttacagacactgaacttgaacttccattgtgatatgaaaacagctatagacaatcataaatgaatgatcatggctatgttttaataaaactttatggacactgagcttgaaaaaaaaaaaaaaaaaa

SEQ ID NO: 11 - protein, 823 aa, Homo sapiens; identical to SEQ ID NO: 1

SEQ ID NO: 12 - RNA, 22 nt, Artificial Sequence
  Synthetic
augcugacauauuuacuagagg

SEQ ID NO: 13 - DNA, 26 bp, Artificial Sequence
  Synthetic
agccgtgtctctcttgatctatgtgt

SEQ ID NO: 14 - DNA, 23 bp, Artificial Sequence
  Synthetic
ggcatttgcttggaatatttagg